(19) United States
(12) Patent Application Publication
(10) Pub. No.: US 2026/0070928 A1
(43) Pub. Date: Mar. 12, 2026

(54) NOVEL CRYSTALLINE FORMS OF (S)-7-OXA-2-AZA-SPIRO[4.5]DECANE-2-CARBOXYLIC ACID [7-(3,6-DIHYDRO-2H-PYRAN-4-YL)-4-METHOXY-THIAZOLO[4,5-C]PYRIDIN-2-YL]-AMIDE AND CO-CRYSTAL FORMS THEREOF

(71) Applicant: Domain Therapeutics S.A., Strassburg (FR)

(72) Inventors: Sylvia Schmidt, Darmstadt (DE); Axel Becker, Darmstadt (DE)

(73) Assignee: Domain Therapeutics S.A., Strassburg (FR)

(21) Appl. No.: 19/100,345

(22) PCT Filed: Jul. 31, 2023
(86) PCT No.: PCT/EP2023/071168

§ 371 Date: Jan. 31, 2025

(30) Foreign Application Priority Data
Aug. 2, 2022 (EP) 22188357.2

(51) Int. Cl. C07D 519/00

(52) U.S. Cl. CPC C07D 519/00

(57) ABSTRACT

A process prepares novel crystalline forms of (S)-7-oxa-2-aza-spiro[4.5]decane-2-carboxylic acid [7-(3,6-dihydro-2H-pyran-4-yl)-4-methoxy-thiazolo[4,5-c]pyridin-2-yl]-amide. The crystalline forms find application in medicaments and pharmaceutical preparations.

The invention relates to novel crystalline forms of (S)-7-Oxa-2-aza-spiro[4,5]decane-2-carboxylic acid [7-(3,6-dihydro-2H-pyran-4-yl)-4-methoxy-thiazolo[4,5-c]pyridin-2-yl]-amide, processes for their preparation, medicaments and pharmaceutical preparations comprising theses forms.

### BACKGROUND OF THE INVENTION

WO 2020/152132 A1 (compound 25) discloses (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide after chiral resolution by HPLC. WO 2020/152132 A1 describes the process for its manufacturing and yields in an amorphous state of the compound. Apart from this, no dedicated crystalline form is mentioned for the compound in WO 2020/152132 A1.

However, if an active substance is intended as an active substance for a medicinal product, the amorphous state being a thermodynamically less stable form in general bears liabilities for chemical and physical stability with a negative impact on the dissolution rate, bioavailability, effectiveness and storage of a drug.

Hence, the aim of the present invention was the development of alternative solid-state forms overcoming these disadvantages.

### SUMMARY OF THE INVENTION

Surprisingly, we have found a novel crystalline anhydrous form, termed A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide.

Furthermore, we have found new hydrate forms of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, namely NF2, NF3, NF4, and NF12.

Additionally, we have found novel anhydrous pure crystalline co-crystal forms of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide/fumaric acid, further on to be named mono fumaric acid co-crystal form A1 and hemi fumaric acid co-crystal form A2, which exhibit improved dissolution behaviour in biorelevant intestinal milieu (FaSSIF) compared to crystalline entities of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide. Besides the above-mentioned co-crystal forms A1 of mono fumaric acid and A2 of hemi fumaric acid, NF1 and NF2 were identified as hemi fumaric acid co-crystal forms.

Furthermore, we surprisingly found novel anhydrous pure crystalline co-crystal forms of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide of 3-hydroxybenzoic acid, further on to be named mono 3-hydroxybenzoic acid co-crystal forms NF1 and NF2.

Finally, we surprisingly have found novel crystalline co-crystal forms of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide and tartaric acid, 1,5-naphthalenedisulfonic acid,_{D}-malic acid and 3,4-dihydroxybenzoic acid, respectively, further on to be named hemi tartaric acid co-crystal forms NF1 and NF2, mono D-malic acid co-crystal form NF1, 1,5-naphthalenedisulfonic acid co-crystal form NF1 and mono 3,4-dihydroxybenzoic acid co-crystal form NF1.

The newly found crystalline forms are thermodynamically more stable than the amorphous state with a positive influence on the dissolution rate, bioavailability, effectiveness and storage when (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide is used for a medicinal product.

Therefore, an embodiment of the present invention are the crystalline forms of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide.

Another embodiment of the present invention is the crystalline anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide.

Yet another embodiment of the present invention are the hydrate forms of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, selected form the group consisting of the hydrate forms NF2, NF3, NF4 and NF12.

Thus, an additional embodiment of the present invention are the crystalline forms of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, selected form the group consisting of
a) the mono fumaric acid co-crystal form A1,
b) the hemi fumaric acid co-crystal form A2,
c) the hemi fumaric acid co-crystal form NF1,
d) the hemi fumaric acid co-crystal form NF2,
e) the mono 3-hydroxybenzoic acid co-crystal form NF1,
f) the mono 3-hydroxybenzoic acid co-crystal form NF2,
g) the hemi tartaric acid co-crystal form NF1,
h) the hemi tartaric acid co-crystal form NF2,
i) the mono D-malic acid co-crystal form NF1,
j) the 1,5-naphthalenedisulfonic acid co-crystal form NF1 and
k) the mono 3,4-dihydroxybenzoic acid co-crystal form NF1.

The novel crystalline anhydrous form A1 shows the following properties in comparison to the amorphous state of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide:
Crystalline morphic form, very good crystallinity
Anhydrous form
Slightly hygroscopic according to Ph. Eur., with no tendency to undergo hydrate formation upon exposure to elevated RH levels

Therefore, another embodiment of the present invention is the crystalline anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the crystalline anhydrous form A1 has the characteristic peaks:

| No. | °2θ (Cu—Kα₁ radiation) ± 0.2° |
|---|---|
| 1 | 7.8 |
| 2 | 9.5 |
| 3 | 10.6 |
| 4 | 11.6 |
| 5 | 12.2 |
| 6 | 15.4 |
| 7 | 16.5 |
| 8 | 18.6 |
| 9 | 19.9 |
| 10 | 20.2 |
| 11 | 21.5 |
| 12 | 22.4 |
| 13 | 23.2 |
| 14 | 23.9 |
| 15 | 24.6 |
| 16 | 25.5 |
| 17 | 28.1 |
| 18 | 28.5 |

Another embodiment of the present invention is the mono fumaric acid co-crystal form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the mono fumaric acid co-crystal form A1 has the characteristic peaks:

| No. | °2θ (Cu—Kα₁ radiation) ± 0.2° |
|---|---|
| 1 | 7.8 |
| 2 | 9.5 |
| 3 | 10.6 |
| 4 | 11.6 |
| 5 | 12.2 |
| 6 | 15.4 |
| 7 | 16.5 |
| 8 | 18.6 |
| 9 | 19.9 |
| 10 | 20.2 |
| 11 | 21.5 |
| 12 | 22.4 |
| 13 | 23.2 |
| 14 | 23.9 |
| 15 | 24.6 |
| 16 | 25.5 |
| 17 | 28.1 |
| 18 | 28.5 |

The novel mono fumaric acid co-crystal form A1 shows the following properties in comparison to the amorphous state of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide:
Crystalline morphic form, very good crystallinity
Anhydrous form
1:1 ratio API:fumaric acid
Slightly hygroscopic according to Ph. Eur., with no tendency to undergo hydrate formation upon exposure to elevated RH levels

Another embodiment of the present invention is the hemi fumaric acid co-crystal form A2 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the hemi fumaric acid co-crystal form A2 has the characteristic peaks:

| No. | °2θ (Cu—Kα₁ radiation) ± 0.2° |
|---|---|
| 1 | 6.6 |
| 2 | 9.7 |
| 3 | 11.5 |
| 4 | 13.4 |
| 5 | 14.1 |
| 6 | 14.7 |
| 7 | 16.5 |
| 8 | 18.0 |
| 9 | 18.5 |
| 10 | 19.4 |
| 11 | 20.0 |
| 12 | 21.4 |
| 13 | 22.9 |
| 14 | 24.1 |
| 15 | 26.8 |

Another embodiment of the present invention is the hemi fumaric acid co-crystal form NF1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the hemi fumaric acid co-crystal form NF1 has the characteristic peaks:

| No. | °2θ (Cu—Kα₁ radiation) ± 0.2° |
|---|---|
| 1 | 6.7 |
| 2 | 8.1 |
| 3 | 10.1 |
| 4 | 12.3 |
| 5 | 16.1 |
| 6 | 16.3 |
| 7 | 16.7 |
| 8 | 19.3 |
| 9 | 21.5 |
| 10 | 22.9 |
| 11 | 25.7 |

Another embodiment of the present invention is the hemi fumaric acid co-crystal form NF2 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the hemi fumaric acid co-crystal form NF2 has the characteristic peaks:

| No. | °2θ (Cu—Kα₁ radiation) ± 0.2° |
|---|---|
| 1 | 6.2 |
| 2 | 8.1 |
| 3 | 9.4 |
| 4 | 10.5 |
| 5 | 11.3 |
| 6 | 14.2 |
| 7 | 14.9 |
| 8 | 16.8 |
| 9 | 17.5 |
| 10 | 19.1 |
| 11 | 20.3 |
| 12 | 20.9 |
| 13 | 21.6 |
| 14 | 22.6 |
| 15 | 24.4 |
| 16 | 25.9 |

Another embodiment of the present invention is the mono 3-hydroxybenzoic acid co-crystal form NF1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the mono 3-hydroxybenzoic acid co-crystal form NF1 has the characteristic peaks:

| No. | °2θ (Cu—Kα₁ radiation) ± 0.2° |
|---|---|
| 1 | 7.3 |
| 2 | 10.6 |
| 3 | 13.9 |
| 4 | 14.5 |
| 5 | 16.1 |
| 6 | 16.6 |
| 7 | 17.4 |
| 8 | 18.1 |
| 9 | 18.7 |
| 10 | 19.4 |
| 11 | 20.0 |
| 12 | 20.8 |
| 13 | 21.3 |
| 14 | 23.1 |
| 15 | 23.5 |
| 16 | 25.1 |
| 17 | 25.7 |
| 18 | 27.2 |
| 19 | 28.1 |

The novel mono 3-hydroxybenzoic acid co-crystal form NF1 shows the following benefits in comparison to the amorphous state of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide:
Crystalline morphic form, very good crystallinity
Anhydrous form
1:1 ratio API: 3-hydroxybenzoic acid
Not hygroscopic according to Ph. Eur., with no tendency to undergo hydrate formation upon exposure to elevated RH levels

Another embodiment of the present invention is the mono 3-hydroxybenzoic acid co-crystal form NF2 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the mono 3-hydroxybenzoic acid co-crystal form NF2 has the characteristic peaks:

| No. | °2θ (Cu—Kα₁ radiation) ± 0.2° |
|---|---|
| 1 | 8.1 |
| 2 | 8.5 |
| 3 | 10.0 |
| 4 | 12.6 |
| 5 | 14.0 |
| 6 | 15.7 |
| 7 | 16.8 |
| 8 | 19.6 |
| 9 | 20.4 |
| 10 | 20.9 |
| 11 | 22.9 |
| 12 | 23.6 |
| 13 | 29.5 |

Another embodiment of the present invention is the mono 3,4-dihydroxybenzoic acid co-crystal form NF1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the mono 3,4-dihydroxybenzoic acid co-crystal form NF1 has the characteristic peaks:

| No. | °2θ (Cu—Kα₁ radiation) ± 0.2° |
|---|---|
| 1 | 7.3 |
| 2 | 10.8 |
| 3 | 13.9 |
| 4 | 15.2 |
| 5 | 15.9 |
| 6 | 16.5 |
| 7 | 17.4 |
| 8 | 18.0 |
| 9 | 18.6 |
| 10 | 19.3 |
| 11 | 20.2 |
| 12 | 21.3 |
| 13 | 23.0 |
| 14 | 23.5 |
| 15 | 25.1 |
| 16 | 25.8 |
| 17 | 27.2 |
| 18 | 28.2 |
| 19 | 29.5 |

Another embodiment of the present invention is the hemi tartaric acid co-crystal form NF1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the hemi tartaric acid co-crystal form NF1 has the characteristic peaks:

| No. | °2θ (Cu—Kα₁ radiation) ± 0.2° |
|---|---|
| 1 | 11.2 |
| 2 | 16.0 |
| 3 | 17.0 |
| 4 | 18.5 |
| 5 | 19.6 |
| 6 | 20.4 |
| 7 | 23.3 |
| 8 | 24.2 |
| 9 | 27.9 |

Another embodiment of the present invention is the hemi tartaric acid co-crystal form NF2 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the hemi tartaric acid co-crystal form NF2 has the characteristic peaks:

| No. | °2θ (Cu—Kα₁ radiation) ± 0.4° |
|---|---|
| 1 | 11.2 |
| 2 | 16.0 |
| 3 | 16.9 |
| 4 | 17.9 |
| 5 | 19.7 |
| 6 | 20.4 |
| 7 | 23.3 |
| 8 | 24.1 |
| 9 | 27.8 |

Another embodiment of the present invention is the 1,5-naphthalenedisulfonic acid co-crystal form NF1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the 1,5-naphthalenedisulfonic acid co-crystal form NF1 has the characteristic peaks:

| No. | °2θ (Cu—Kα₁ radiation) ± 0.4° |
|---|---|
| 1 | 6.5 |
| 2 | 8.4 |
| 3 | 10.1 |
| 4 | 10.4 |
| 5 | 12.0 |
| 6 | 13.1 |
| 7 | 14.8 |
| 8 | 15.3 |
| 9 | 16.9 |
| 10 | 17.7 |
| 11 | 18.2 |
| 12 | 18.6 |
| 13 | 19.4 |
| 14 | 20.3 |
| 15 | 21.0 |
| 16 | 21.8 |
| 17 | 22.2 |
| 18 | 22.8 |
| 19 | 25.0 |

Another embodiment of the present invention is the mono D-malic acid co-crystal form NF1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, wherein the mono_{D}-malic acid co-crystal form NF1 has the characteristic peaks:

| No. | °2θ (Cu—Kα₁ radiation) ± 0.2° |
|---|---|
| 1 | 17.0 |
| 2 | 19.6 |
| 3 | 20.4 |
| 4 | 21.6 |
| 5 | 23.4 |
| 6 | 28.1 |

All forms can be characterized according to standard methods which can be found in e.g.:
Rolf Hilfiker, ‘Polymorphism in the Pharmaceutical Industry’, Wiley-VCH. Weinheim 2006 (Chapter 6: X-Ray Diffraction, Chapter 6: Vibrational Spectroscopy, Chapter 3: Thermal Analysis, Chapter 9: Water Vapour Sorption, and references therein)
H. G. Brittain, ‘Polymorphism in Pharmaceutical Solids, Vol. 95, Marcel Dekker Inc., New York 1999 (Chapter 6 and references therein)

The invention also relates to (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide containing one or more of the co-crystal forms according to the present invention.

A further embodiment according to the present invention is (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide essentially consisting of one or more of the co-crystal forms according to the present invention.

The co-crystal forms according to the present invention are prepared by suspending (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide in an organic solvent, preferably acetone, at elevated temperature and after adding the equivalent amount of a co-former a cooling crystallization is started and finally the residue is separated off and dried.

Thus, another embodiment of the present invention is a method for the preparation of the co-crystal forms according to the present invention, characterized in that (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide is suspended in an organic solvent at elevated temperature, the equivalent amount of a co-former is added and a cooling crystallization is carried out.

As already described, the crystalline forms according to the present invention are thermodynamically stable and therefore particularly suitable as a medicinal product. As described in WO 2020/152132 A1 (S)-7-Oxa-2-aza-spiro[4,5]decane-2-carboxylic acid [7-(3,6-dihydro-2H-pyran-4-yl)-4-methoxy-thiazolo[4,5-c]pyridin-2-yl]-amide can be used for the treatment and/or prophylaxis of physiological and/or pathophysiological states, selected from the group consisting of hyperproliferative and infectious diseases and disorders and in particular for the treatment and/or prophylaxis of cancer.

The active ingredient can of course also be used as a mixture of the crystalline forms according to the present invention, with more than 10% by weight, 20% by weight, 30% by weight, 40% by weight, 50% by weight, 60% by weight Wt.-%, 70 wt .-%, 80 wt.-% or 90 wt.-% of the crystalline forms according to the present invention can be contained. Mixtures with more than 70% by weight, particularly preferably with more than 80% by weight and particularly preferably with more than 90% by weight of the crystalline forms according to the present invention are preferred according to the invention.

The invention therefore also relates to a medicament comprising one or more of the crystalline forms according to the present invention or mixtures thereof in all ratios for use in the treatment and/or prophylaxis of cancer.

Another embodiment of the present invention is the use of one or more of the crystalline forms according to the present invention or mixtures thereof in all ratios for the preparation of a medicament for use in the treatment and/or prophylaxis of cancer.

Yet another embodiment of the present invention is a method of treatment and/or prophylaxis of cancer, wherein one or more of the crystalline forms according to the present invention or mixtures thereof are administered to a person in need thereof.

Additionally, the invention relates to a pharmaceutical preparation containing one or more of the crystalline forms according to the present invention or mixtures thereof in all ratios and optionally further excipients and/or adjuvants.

Another embodiment of the present invention is a process for the preparation of a pharmaceutical preparation, characterised in that one or more of the crystalline forms according to the present invention or mixtures thereof in all ratios are brought into a suitable dosage form together with a solid, liquid or semi-liquid excipient or adjuvant.

Even without further embodiments, it is assumed that a person skilled in the art will be able to use the above description in the broadest scope. The preferred embodiments should therefore merely be regarded as descriptive disclosure which is absolutely not limiting in any way.

All the references cited herein are incorporated by reference in the disclosure of the invention hereby.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

### Abbreviations

2,4-DHBA: 2,4-dihydroxybenzoic acid
3,4-DHBA: 3,4-dihydroxybenzoic acid
3-HBA: 3-hydroxybenzoic acid
DSC: differential scanning calorimetry
TGA: thermogravimetric analysis
SGF: simulated gastric fluid
FeSSIF: fed state simulated intestinal fluid
FaSSIF: fasted state simulated intestinal fluid

### Example 1—Preparation of the amorphous state of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

WO 2020/152132 A1 describes the chiral separation of an intermediate by chiral HPLC yield in amorphous material of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide.

A powder X-ray diffraction pattern has been obtained by standard techniques as described in the European Pharmacopeia 6th Edition chapter 2.9.33 and is characterised by the X-ray powder diffractogram (monochromatic Cu-Kα₁ radiation, λ=1.5406, Stoe StadiP 611 KL transmission diffractometer) shown in FIG. 1.

The amorphous state is characterised by the following physical properties: Thermal behaviour shows no significant enthalpic events and TGA a total weight loss up to 150° C. of 2.9% (w/w). The DSC and TGA profiles are displayed in FIGS. 2A and 2B. A DSC scan of the amorphous state was acquired on a Mettler-Toledo DSC1 with a heating rate of 5 K/min, using nitrogen purge gas at 50 mL/min. A TGA scan of the amorphous state was acquired on a Mettler-Toledo TGA 851 with a heating rate of 5 K/min, using nitrogen purge gas at 50 mL/min (DSC and TGA scan of amorphous state see FIGS. 2A and 2B).

The water vapour sorption behaviour reveals water uptake levels >22% (w/w) in the full relative humidity (RH) range 0-98% RH. The amorphous state can be classified as hygroscopic according to Ph. Eur. criteria (section 5.11.) and it shows tendencies for deliquescence at humidity levels >90% RH. The water vapour sorption isotherm (25° C.) is displayed in FIG. 3 (Water Vapour Sorption Isotherm (25° C.) of amorphous state). The water vapour sorption isotherm was acquired on a DVS intrinsic system from SMS.

### Example 2—Process for the preparation of the crystalline anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

Lab scale:

Approximately 210 mg of the amorphous state of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were stirred in 2 mL acetone at 50° C. for 2 hours. Afterwards, the suspension was centrifuged and dried for 12 hours at 50° C. under N₂-flux.

Gramm scale:

Approximately 1.9 g of the amorphous state of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were resolved from 20 mL acetone, stored over night at 0° C., separated and dried. Afterwards, the suspension was centrifuged and dried for 90 hours at 50° C. and <10 mbar.

A powder X-ray diffraction pattern of A1 has been obtained by standard techniques as described in the European Pharmacopeia 6th Edition chapter 2.9.33 and is characterised by the powder X-ray diffractogram (monochromatic Cu-Kα₁ radiation, λ=1.5406 Å, Stoe StadiP 611 KL transmission diffractometer) shown in FIG. 4. A powder X-ray peak list of the A1 form of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide is shown in table 1.

TABLE 1
Powder X-ray peak list of the crystalline anhydrous form A1

| No. | °2θ (Cu—Kα₁ radiation) ± 0.2° |
|---|---|
| 1 | 7.8 |
| 2 | 9.5 |
| 3 | 10.6 |
| 4 | 11.6 |
| 5 | 12.2 |
| 6 | 15.4 |
| 7 | 16.5 |
| 8 | 18.6 |
| 9 | 19.9 |
| 10 | 20.2 |
| 11 | 21.5 |
| 12 | 22.4 |
| 13 | 23.2 |
| 14 | 23.9 |
| 15 | 24.6 |
| 16 | 25.5 |
| 17 | 28.1 |
| 18 | 28.5 |

Single crystal x-ray structure data were obtained on the A1 form as well (Oxford Diffraction Supernova Single Crystal X-ray Diffractometer with graphite monochromator and CCD detector) at 298 K (see FIG. 5).

A1 form crystallises in the non-centrosymmetric, monoclinic space group P21 with the lattice parameters a=9.1±0.1 Å, b=22.5±0.1 Å, c=10.4±0.1 Å, and β=103.5±0.5° (with α=γ=90°). From the single crystal structure, it is obvious that form A1 represents an anhydrous form.

The A1 form is characterised by the following physical properties:

The thermal behaviour of the A1 form shows melting (˜213° C.) overlapping with a TGA step, which is due to thermal release of strongly bounded residual solvents in agglomerates. The DSC and TGA profiles are displayed in FIGS. 6A and 6B. A DSC scan of the A1 form was acquired on a Mettler-Toledo DSC1 with a heating rate of 5 K/min, using nitrogen purge gas at 50 mL/min. A TGA scan of the A1 form was acquired on a Mettler-Toledo TGA 851 with a heating rate of 5 K/min, using nitrogen purge gas at 50 mL/min.

The water vapour sorption behaviour of the A1 form reveals very small water uptake levels <1% (w/w) in the full relative humidity (RH) range 0-98% RH. The A1 form can be classified as slightly hygroscopic according to Ph. Eur. criteria (section 5.11.). The water vapor sorption isotherm (25° C.) of form A1 is displayed in FIG. 7.

The water vapour sorption isotherm was acquired on a DVS Intrinsic system from SMS.

### Example 3—Process for the preparation of the hydrate form NF2 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

Approximately 17 mg of the amorphous state of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were stirred in 500 μL water at room temperature for approx.7 days. The solid residue was centrifuged and not dried.

A powder X-ray diffraction pattern of the hydrate form NF2 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 8.

### Example 4—Process for the preparation of the hydrate form NF3 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

Approximately 17 mg of the amorphous state of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were stirred in 500 μL water at room temperature for approximately 7 days. The solid residue was centrifuged and dried at ambient conditions.

A powder X-ray diffraction pattern of the hydrate form NF3 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 9.

### Example 5—Process for the preparation of the hydrate form NF4 of of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

Approximately 5 mg of the hydrate form NF4 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were suspended in 1 mL FeSSIF [pH 5.0] at 37° C. for 24 hours. The solid/liquid separation was carried out by centrifugation.

A Powder X-ray diffraction pattern of the hydrate form NF4 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 10.

### Example 6—Process for the preparation of the hydrate form NF12 of of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

Approximately 5 mg of the hydrate form NF12 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were suspended in 1 mL USP phosphate buffer[pH 3.2] at 37° C. for 24 hours. The solid/liquid separation was carried out by centrifugation.

A powder X-ray diffraction pattern of the hydrate form NF12 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 11.

### Example 7—Processes for co-crystal screening for 8-Oxa-2-azaspiro[4,5]decane-2-carboxylic acid (4-methoxy-7-phenylthiazolo[4,5-c]pyridin-2-yl)-amide

A co-crystal screening was performed for 8-Oxa-2-azaspiro[4,5]decane-2-carboxylic acid (4-methoxy-7-phenylthiazolo[4,5-c]pyridin-2-yl)-amide (compound 53 in WO 2019/025099). A broad range of experiment types (e.g. co-melting, grinding, cooling crystallisation) was used, however, surprisingly yielded in 1:1 ratio only with very few co-formers, namely 3-hydroxybenzoic acid, tartaric acid, and 2,4-dihydroxybenzoic acid, respectively. For larger scale, most promising results were observed in cooling crystallization trials. Since 8-Oxa-2-azaspiro[4,5]decane-2-carboxylic acid (4-methoxy-7-phenylthiazolo[4,5-c]pyridin-2-yl)-amide is not the subject of the invention, no detailed physico-chemical information are included.

Based on the outcome of the co-crystal screening, for (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide only cooling crystallization, partly followed by vapour diffusion experiments, were applied, since both compounds are structurally similar.

In the following table 3, experiment types and used co-formers for 8-Oxa-2-aza-spiro[4,5]decane-2-carboxylic acid (4-methoxy-7-phenylthiazolo[4,5-c]pyridin-2-yl)-amide are described.

TABLE 2

| | | | Cooling |
|---|---|---|---|
| Co-former | grinding | Co-melting | crystallisation |
| Fumaric acid | x | x | ✓ |
| tert-Butylhydrochinone | x | x | x |
| 2,4-DHBA | x | ✓ | ✓ |
| 3,4-DHBA | x | x | ✓ |
| L-Tartaric acid | x | x | ✓ |
| Citric acid | x | x | ✓ |
| 3-hydroxybenzoic-acid | x | x | ✓ |
| 2-oxopentanedioic-acid | x | x | x |
| Sulfuric acid | x | x | x |

### Example 8—Processes for co-crystal screening for S)-7-Oxa-2-aza-spiro[4,5]decane-2-carboxylic acid [7-(3,6-dihydro2H-pyran-4-yl)-4-methoxy-thiazolo[4, 5-c]pyridin-2-yl]-amide

Based on knowledge in experiments of 8-Oxa-2-azaspiro[4,5]decane-2-carboxylic acid (4-methoxy-7-phenylthiazolo[4,5-c]pyridin-2-yl)-amide and structural similarity between 8-Oxa-2-azaspiro[4,5]decane-2-carboxylic acid (4-methoxy-7-phenylthiazolo[4,5-c]pyridin-2-yl)-amide and (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide, the successful experiment type ‘cooling crystallisation’ was further applied also for (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide. In few experiments without any solid residue, an additional experiment step (vapour diffusion) was carried out.

TABLE 3

| | Cooling |
|---|---|
| | crystallisation/ |
| Co-former | Vapour diffusion |
| Fumaric acid | ✓ |
| 1,2-Ethanedisulfonic acid | x |
| 1,5-Naphthalenedisulfonic acid | ✓ |
| tert-Butylhydrochinone | x |
| 2,4-DHBA | x |
| 3,4-DHBA | ✓ |
| D-Tartaric acid | ✓ |
| L-Tartaric acid | ✓ |
| Citric acid | x |
| 3-hydroxybenzoic-acid | ✓ |
| L-Malic acid | x |
| D-Malic acid | ✓ |
| Sulfuric acid | x |

### Example 9—Preparation processes for novel mono fumaric acid co-crystal form A1

Lab scale:

Approximately 19 mg of the anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 0.7 mL acetone at 50° C.

Approximately 6 mg of fumaric acid were added and a cooling ramp was started three times (50-5° C. with 0.1 K/min). The suspension was centrifuged for liquid/solid separation and afterwards, the solid was dried at room temperature.

kg-scale:

Approximately 4.3 kg of anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide and approximately 1.3 kg of fumaric acid were suspended in 15 V acetone at ˜25° C. for at least 10 min with medium stirrer speed. The suspension was heated up to ˜65° C. in 1 hour. Afterwards, a cooling ramp was started (˜65-˜10° C. in 7 hours). The suspension was stirred at ˜10° C. for at least 4 hours and filtered with suction for liquid/solid separation and finally washed with 1 V of acetone. The solid was dried with vacuum at least for 8 hours at ˜70° C.

A powder X-ray diffraction pattern of the mono fumaric acid co-crystal form A1 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 12.

NMR data for the mono fumaric acid co-crystal form A1:

¹H NMR (700 MHz, DMSO-d6) δ 13.18-13.06 (m, 2H), 11.39-11.30 (m, 1H), 7.94 (s, 1H), 6.62 (s, 2H), 6.25-6.23 (m, 1H), 4.29 (q, J=2.8 Hz, 2H), 3.99 (s, 3H), 3.87 (t, J=5.4 Hz, 2H), 3.66-3.42 (m, 5H), 3.40 (d, J=11.3 Hz, 1H), 3.31-3.28 (m, 1H), 3.26-3.08 (m, 1H), 2.57-2.53 (m, 2H), 1.90-1.74 (m, 1H), 1.74-1.47 (m, 5H).

TABLE 4
Powder X-ray peak list of the mono
fumaric acid co-crystal form A1

| No. | °2θ (Cu—Kα₁ radiation) ± 0.2° |
|---|---|
| 1 | 7.2 |
| 2 | 8.0 |
| 3 | 11.7 |
| 4 | 13.3 |
| 5 | 14.0 |
| 6 | 14.9 |
| 7 | 15.3 |
| 8 | 15.7 |
| 9 | 16.2 |
| 10 | 17.8 |
| 11 | 18.1 |
| 12 | 20.2 |
| 13 | 21.5 |
| 14 | 23.0 |
| 15 | 24.3 |
| 16 | 25.0 |
| 17 | 27.4 |

Single crystal X-ray structure data were obtained on the mono fumaric acid co-crystal form A1 (Oxford Diffraction Supernova Single Crystal X-ray Diffractometer with Graphite monochromator and CCD Detector) as shown in FIG. 13.

The mono fumaric acid co-crystal form A1 crystallises in the monoclinic space group P2₁ with the lattice parameters a=8.9±0.1 Å, b=24.1±0.1 Å, c=12.5±0.1 Å, and β=101.3±0.5° (with α=γ=90°). From the single crystal structure, it is obvious that the mono fumaric acid co-crystal form A1 represents a mono fumaric acid co-crystal anhydrous form.

The mono fumaric acid co-crystal form A1 is characterised by the following physical properties:
1 eq. Fumaric acid (determined by NMR)
Thermal behaviour of the mono fumaric acid co-crystal form A1 form shows an overlapping melting/decomposition process (>207° C.) with only small weight losses (<1% (w/w) prior melting/decomposition). The DSC and TGA profiles are displayed in FIGS. 14A and 14B. The DSC scan of mono fumaric acid co-crystal form A1 was acquired on a Mettler-Toledo DSC1 with a heating rate of 5 K/min, using nitrogen purge gas at 50 mL/min. The TGA scan of mono fumaric acid co-crystal form A1 was acquired on a Mettler-Toledo TGA 851 with a heating rate of 5 K/min, using nitrogen purge gas at 50 mL/min.
Water Vapour Sorption behaviour of mono fumaric acid co-crystal form A1 form reveals very small water uptake levels <3% (w/w) in the full relative humidity (RH) range 0-98% RH. The mono fumaric acid co-crystal form A1 can be classified as slightly hygroscopic according to Ph. Eur. criteria (section 5.11.). The water vapor sorption isotherm (25° C.) of the mono fumaric acid co-crystal form A1 is displayed in FIG. 15. The water vapour sorption isotherm was acquired on a DVS intrinsic system from SMS.
The concentration levels of the mono fumaric acid co-crystal form A1 were determined at 37° C. after 15 min and 30 min in non-sink-dissolution experiments.

TABLE 5
Dissolution levels of the mono fumaric acid co-crystal form A1

| | |
|---|---|
| Dissolution | Dissolution levels [μg/mL] after |

| medium | 15 min | 30 min |
|---|---|---|
| SGF [pH 1.2] | 331 | 385 |
| FeSSIF [pH 5.0] | 375 | 462 |
| FaSSIF [pH 6.5] | 278 | 360 |

In the following, few examples are described where formation of mono fumaric acid co-crystal form A1 failed.

TABLE 6
Preparation process

| | |
|---|---|
| Approximately 20 mg of anhydrous form A1 of | no solid residue |
| compound 25 were dispersed in 500 μL | |
| tetrahydrofuran at 50° C. | |
| Approximately 6 mg of fumaric acid were added | |
| and a cooling ramp was started three times (50 - | |
| 5° C. with 0.1 K/min). No precipitation occurred and | |
| the clear solution was stored for Approximately 4 | |
| months under n-pentane atmosphere. No | |
| precipitation was observed. | |
| Approximately 50 mg of anhydrous form A1 of | obtained as |
| compound 25 were dispersed in 2.5 mL | NF2 of hemi fumaric |
| methanol/ethyl acetate (mixture 1:1) at 50° C. | acid co-crystal form of |
| Approximately 15 mg of fumaric acid were added | (S)-7-Oxa-2-aza- |
| and a cooling ramp was started three times (50 - | spiro[4.5]decane-2- |
| 5° C. with 0.1 K/min). Precipitation was observed at | carboxylic acid [7-(3,6- |
| 5° C. | dihydro2H-pyran-4-yl)-4- |
| | methoxy-thiazolo[4,5- |
| | c]pyridin-2-yl]-amide |
| Approximately 65 mg of anhydrous form A1 of | obtained as |
| compound 25 were dispersed in 2.5 mL | NF1 of hemi fumaric |
| methanol/ethyl acetate (mixture 20:80) at 50° C. | acid co-crystal form of |
| Approximately 19 mg of fumaric acid were added | (S)-7-Oxa-2-aza- |
| and a cooling ramp was started three times (50 - | spiro[4.5]decane-2- |
| 5° C. with 0.1 K/min). Precipitation was observed at | carboxylic acid [7-(3,6- |
| 5° C. | dihydro2H-pyran-4-yl)-4- |
| | methoxy-thiazolo[4,5- |
| | c]pyridin-2-yl]-amide |
| Approximately 60 mg of anhydrous form A1 of | obtained as |
| compound 25 were dispersed in 2.5 mL | NF2 of hemi fumaric |
| acetone/methanol (mixture 1:1) at 50° C. | acid co-crystal form of |
| Approximately 17 mg of fumaric acid were added | (S)-7-Oxa-2-aza- |
| and a cooling ramp was started three times (50 - | spiro[4.5]decane-2- |
| 5° C. with 0.1 K/min). Precipitation was observed at | carboxylic acid [7-(3,6- |
| 5° C. | dihydro2H-pyran-4-yl)-4- |
| | methoxy-thiazolo[4,5- |
| | c]pyridin-2-yl]-amide 5 |
| Approximately 23 mg of anhydrous form A1 of | obtained as |
| compound 25 were dissolved in 1.6 mL 2- | NF1 of hemi fumaric |
| methyltetrahydrofuran/acetonitrile (mixture 9:1) at | acid co-crystal form of |
| 50° C. | (S)-7-Oxa-2-aza- |
| Approximately 7 mg of fumaric acid were | spiro[4.5]decane-2- |
| dissolved in 0.5 mL 2- | carboxylic acid [7-(3,6- |
| methyltetrahydrofuran/acetonitrile (mixture 9:1) | dihydro2H-pyran-4-yl)-4- |
| and added into the API-solution. A cooling ramp | methoxy-thiazolo[4,5- |
| was started (50 - 25° C. with 0.1 K/min). | c]pyridin-2-yl]-amide |
| Precipitation was observed at 25° C. | |

### Example 10—Preparation Process for Novel Hemi Fumaric Acid Co-Crystal Form A2

### Preparation Process

Approximately 140 mg of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 8 mL isopropanol at 50° C. and a cooling ramp was started (50-5° C. with 0.1 K/min). Precipitation was observed at 5° C.

NMR data for the hemi fumaric acid co-crystal form A2

¹H NMR (500 MHz, DMSO-d6) δ 13.31-12.94 (m, 1H), 11.44-11.23 (m, 1H), 7.94 (s, 1H), 6.61 (s, 1H), 6.26-6.22 (m, 1H), 4.29 (q, J=2.8 Hz, 2H), 3.99 (s, 3H), 3.87 (t, J=5.4 Hz, 2H), 3.68-3.40 (m, 5H), 3.40 (d, J=11.2 Hz, 1H), 3.35-3.30 (m, 1H), 3.26-3.08 (m, 1H), 2.58-2.53 (m, 2H), 1.90-1.73 (m, 1H), 1.73-1.46 (m, 5H)

A powder X-ray diffraction pattern of the hemi fumaric acid co-crystal form A2 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 16.

TABLE 7
Powder X-ray peak list of the hemi
fumaric acid co-crystal form A2

| No. | °2θ (Cu—Kα₁ radiation) ± 0.2° |
|---|---|
| 1 | 6.6 |
| 2 | 9.7 |
| 3 | 11.5 |
| 4 | 13.4 |
| 5 | 14.1 |
| 6 | 14.7 |
| 7 | 16.5 |
| 8 | 18.0 |
| 9 | 18.5 |
| 10 | 19.4 |
| 11 | 20.0 |
| 12 | 21.4 |
| 13 | 22.9 |
| 14 | 24.1 |
| 15 | 26.8 |

The single crystal X-ray structure data were obtained on the hemi fumaric acid co-crystal form A2 as well (Oxford Diffraction Supernova Single Crystal X-ray Diffractometer with Graphite monochromator and CCD Detector), see FIG. 17.

The temi fumaric acid co-crystal form A2 crystallises in the triclinic space group P1 with the lattice parameters a=9.96±0.1 Å, b=15.7±0.1 Å, c=17.2±0.1 Å, and α=66.5±0.5°, β=77.5±0.5° and γ=76.9±0.5°. From the single crystal structure, it is obvious that the hemi fumaric acid co-crystal form A2 represents an anhydrous form.

The hemi fumaric acid co-crystal form A2 is characterised by the following physical properties:
0.5 eq. Fumaric acid (determined by NMR)
The thermal behaviour of the hemi fumaric acid co-crystal form A2 shows an overlapping melting/decomposition process (>207° C.) with only small weight losses (<1% (w/w) prior melting/decomposition). The DSC and TGA profiles are displayed in FIGS. 18A and 18B. The DSC scan of the hemi fumaric acid co-crystal form A2 was acquired on a Mettler-Toledo DSC1 with a heating rate of 5 K/min, using nitrogen purge gas at 50 mL/min. The TGA scan of hemi fumaric acid co-crystal form A2 was acquired on a Mettler-Toledo TGA 851 with a heating rate of 5 K/min, using nitrogen purge gas at 50 mL/min.
The water vapour sorption behaviour of the hemi fumaric acid co-crystal form A2 reveals very small water uptake levels ≤0.7% (w/w) in the relative humidity (RH) range 0-80% RH. The hemi fumaric acid co-crystal form A2 can be classified as slightly hygroscopic according to Ph. Eur. Criteria (section 5.11.). The water vapor sorption isotherm (25° C.) of the hemi fumaric acid co-crystal form A2 is displayed in FIG. 19. The water vapour sorption isotherm was acquired on a DVS intrinsic system from SMS.
The concentration levels of the hemi fumaric acid co-crystal form A2 were determined at 37° C. after 15 min and 30 min in non-sink-dissolution experiments.

TABLE 8
Dissolution levels of the hemi fumaric acid co-crystal form A2

| | |
|---|---|
| Dissolution | Dissolution levels [μg/mL] after |

| medium | 15 min | 30 min |
|---|---|---|
| SGF [pH 1.2] | 206 | 292 |
| FeSSIF [pH 5.0] | 651 | 899 |
| FaSSIF [pH 6.5] | 317 | 492 |

### Example 11—Hemi fumaric acid co-crystal form NF1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

### Preparation Process for the Novel Hemi Fumaric Acid Co-Crystal Form NF1

Approximately 15 mg of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 500 μL 1,4-dioxane at 25° C. and stirred for 5 days. For liquid/solid separation, the suspension was centrifuged and the solid was dried at room temperature.

NMR data for the hemi fumaric acid co-crystal form NF1

¹H NMR (500 MHz, DMSO-d₆) δ 11.36-11.29 (m, 1H), 7.94 (s, 1H), 6.25-6.22 (m, 1H), 4.29 (q, J=2.8 Hz, 2H), 3.99 (s, 3H), 3.87 (t, J=5.4 Hz, 2H), 3.67-3.38 (m, 6H), 3.34-3.28 (m, 2H), 2.58-2.53 (m, 2H), 1.90-1.75 (m, 1H), 1.73-1.47 (m, 5H).

A powder X-ray diffraction pattern of the hemi fumaric acid co-crystal form NF1 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 20.

TABLE 9
Powder X-ray peak list of the hemi
fumaric acid co-crystal form NF1

| No. | °2θ (Cu—Kα₁ radiation) ± 0.2° |
|---|---|
| 1 | 6.7 |
| 2 | 8.1 |
| 3 | 10.1 |
| 4 | 12.3 |
| 5 | 16.1 |
| 6 | 16.3 |
| 7 | 16.7 |
| 8 | 19.3 |
| 9 | 21.5 |
| 10 | 22.9 |
| 11 | 25.7 |

The hemi fumaric acid co-crystal form form NF1 is characterised by the following physical properties:
0.5 eq. Fumaric acid (determined by NMR)
The thermal behaviour of the hemi fumaric acid co-crystal form NF1 shows a small endothermic event prior melting at ˜220° C. with only small weight losses (<1% (w/w) prior melting). The DSC and TGA profiles are displayed in FIGS. 21A and 21B. The DSC scan of hemi fumaric acid co-crystal form NF1 was acquired on a Mettler-Toledo DSC1 with a heating rate of 10 K/min, using nitrogen purge gas at 50 mL/min. The TGA scan of hemi fumaric acid co-crystal form NF1 was acquired on a Mettler-Toledo TGA 851 with a heating rate of 10 K/min, using nitrogen purge gas at 50 mL/min.

### Example 12—Hemi fumaric acid co-crystal form NF2 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

### Preparation Process for the Novel Hemi Fumaric Acid Co-Crystal/Salt Form NF2

Approximately 60 mg of the anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 2.5 mL acetone/methanol (mixture 1:1) at 50° C. Approximately 17 mg of fumaric acid were added and a cooling ramp was started three times (50-5° C. with 0.1 K/min). Precipitation was observed at 5° C.

NMR data for the hemi fumaric acid co-crystal/salt form NF2

¹H NMR (500 MHz, DMSO-d₆) δ 11.41-11.23 (m, 1H), 7.93 (s, 1H), 6.25-6.22 (m, 1H), 4.29 (q, J=2.8 Hz, 2H), 3.99 (s, 3H), 3.87 (t, J=5.4 Hz, 2H), 3.67-3.10 (m, 6H), 3.31 (d, J=11.3 Hz, 2H), 2.58-2.52 (m, 2H), 1.90-1.74 (m, 1H), 1.73-1.46 (m, 5H).

A powder X-ray diffraction pattern of the hemi fumaric acid co-crystal form NF2 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 22.

TABLE 10
Powder X-ray peak list of the hemi
fumaric acid co-crystal form NF2

| No. | °2θ (Cu—Kα₁ radiation) ± 0.2° |
|---|---|
| 1 | 6.2 |
| 2 | 8.1 |
| 3 | 9.4 |
| 4 | 10.5 |
| 5 | 11.3 |
| 6 | 14.2 |
| 7 | 14.9 |
| 8 | 16.8 |
| 9 | 17.5 |
| 10 | 19.1 |
| 11 | 20.3 |
| 12 | 20.9 |
| 13 | 21.6 |
| 14 | 22.6 |
| 15 | 24.4 |
| 16 | 25.9 |

The hemi fumaric acid co-crystal form NF2 is characterised by the following physical properties:
0.5 eq. Fumaric acid (determined by NMR)
The thermal behaviour of the hemi fumaric acid co-crystal form NF2 shows an overlapping melting/decomposition process (>191° C.) with only small weight losses (<1% (w/w) prior melting/decomposition). The DSC and TGA profiles are displayed in FIGS. 23A and 23B. The DSC scan of the hemi fumaric acid co-crystal form NF2 was acquired on a Mettler-Toledo DSC1 with a heating rate of 5 K/min, using nitrogen purge gas at 50 mL/min. The TGA scan of the hemi fumaric acid co-crystal form NF2 was acquired on a Mettler-Toledo TGA 851 with a heating rate of 5 K/min, using nitrogen purge gas at 50 mL/min.

### Example 13—3-Hydroxybenzoic acid co-crystal form NF1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-aide

### Preparation Process for the Novel Mono 3-Hydroxybenzoic Acid Co-Crystal Form NF1

Approximately 16 mg of the anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 0.7 mL acetone at 50° C. Approximately 7 mg of 3-hydroxybenzoic acid were added and a cooling ramp was started three times (50-5° C. with 0.1 K/min). Precipitation was observed at 5° C.

NMR data for the mono 3-hydroxybenzoic acid co-crystal form NF1

¹H NMR (500 MHz, DMSO-d6) δ 12.79-12.68 (m, 1H), 11.35-11.26 (m, 1H), 9.67 (s, 1H), 7.94 (s, 1H), 7.39-7.35 (m, 1H), 7.34-7.32 (m, 1H), 7.28 (t, J=7.9 Hz, 1H), 7.01-6.97 (m, 1H), 6.26-6.22 (m, 1H), 4.29 (q, J=2.8 Hz, 2H), 4.00 (s, 3H), 3.87 (t, J=5.4 Hz, 2H), 3.67-3.42 (m, 5H), 3.41 (d, J=11.2 Hz, 1H), 3.32 (d, J=11.2 Hz, 1H), 3.26-3.12 (m, 1H), 2.58-2.52 (m, 2H), 1.90-1.74 (m, 1H), 1.74-1.46 (m, 5H)

A powder X-ray diffraction pattern of the mono 3-hydroxybenzoic acid co-crystal form NF1 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 24.

TABLE 11
Powder X-ray peak list of the mono 3-hydroxybenzoic
acid co-crystal form NF1

| No. | °2θ (Cu—Kα₁ radiation) ± 0.2° |
|---|---|
| 1 | 7.3 |
| 2 | 10.6 |
| 3 | 13.9 |
| 4 | 14.5 |
| 5 | 16.1 |
| 6 | 16.6 |
| 7 | 17.4 |
| 8 | 18.1 |
| 9 | 18.7 |
| 10 | 19.4 |
| 11 | 20.0 |
| 12 | 20.8 |
| 13 | 21.3 |
| 14 | 23.1 |
| 15 | 23.5 |
| 16 | 25.1 |
| 17 | 25.7 |
| 18 | 27.2 |
| 19 | 28.1 |

The mono 3-hydroxybenzoic acid co-crystal form NF1 is characterised by the following physical properties:
3-hydroxybenzoic acid content (determined by ¹H-NMR spectroscopy) reveals 1 eq. 3-hydroxybenzoic acid
The thermal behaviour of the mono 3-hydroxybenzoic acid co-crystal form NF1 shows small weight loss steps prior melting (Δm @116° C.: 0.4% (w/w), Δm 116-175° C.: 0.8% (w/w)) and strong weight loss in the TGA profile >175° C. This can be assigned to release of residual solvent followed by decomposition processes of the mono 3-hydroxybenzoic acid co-crystal form NF1. The DSC and TGA profiles are displayed in FIGS. 25A and 25B. The DSC scan of the mono 3-hydroxybenzoic acid co-crystal form NF1 was acquired on a Mettler-Toledo DSC1 with a heating rate of 5 K/min, using nitrogen purge gas at 50 mL/min. The TGA scan of the mono 3-hydroxybenzoic acid co-crystal form NF1 was acquired on a Mettler-Toledo TGA 851 with a heating rate of 5 K/min, using nitrogen purge gas at 50 mL/min.
The water vapour sorption isotherm (25° C.) of the mono 3-hydroxybenzoic acid co-crystal form NF1 is shown in FIG. 26.

### Example 14—3-Hydroxybenzoic co-crystal form NF2 of S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

### Preparation Process for the Mono 3-Hydroxybenzoic Acid Co-Crystal Form NF2

Approximately 13 mg of the anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 0.5 mL tetrahydrofuran at 50° C. Approximately 5 mg of 3-hydroxybenzoic acid were added and a cooling ramp was started three times (50-5° C. with 0.1 K/min). Since no precipitation was observed at 5° C., a vapour diffusion experiment was added by using n-pentane as anti-solvent. After 4 months, few particles were obtained.

NMR data for the mono 3-hydroxybenzoic acid co-crystal form NF2

¹H NMR (500 MHz, DMSO-d6) δ 13.02-12.37 (m, 1H), 11.37-11.21 (m, 1H), 9.73-9.62 (m, 1H), 7.93 (s, 1H), 7.38-7.35 (m, 1H), 7.34-7.32 (m, 1H), 7.28 (t, J=7.8 Hz, 1H), 7.00-6.97 (m, 1H), 6.25-6.22 (m, 1H), 4.29 (q, J=2.7 Hz, 2H), 3.99 (s, 3H), 3.87 (t, J=5.4 Hz, 2H), 3.67-3.43 (m, 5H), 3.40 (d, J=11.4 Hz, 1H), 3.32 (d, J=11.5 Hz, 1H), 3.26-3.10 (m, 1H), 2.58-2.52 (m, 2H), 1.89-1.78 (m, 1H), 1.72-1.47 (m, 5H).

A powder X-ray diffraction pattern of mono 3-hydroxybenzoic co-crystal form NF2 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 27.

TABLE 12
Powder X-ray peak list of mono 3-
hydroxybenzoic co-crystal form NF2

| No. | °2θ (Cu—Kα₁ radiation) ± 0.2° |
|---|---|
| 1 | 8.1 |
| 2 | 8.5 |
| 3 | 10.0 |
| 4 | 12.6 |
| 5 | 14.0 |
| 6 | 15.7 |
| 7 | 16.8 |
| 8 | 19.6 |
| 9 | 20.4 |
| 10 | 20.9 |
| 11 | 22.9 |
| 12 | 23.6 |
| 13 | 29.5 |

The mono 3-hydroxybenzoic co-crystal form NF2 is characterised by the following physical properties:
3-Hydroxybenzoic acid content (determined by ¹H-NMR spectroscopy) reveals 1 eq. 3-hydroxybenzoic acid

### Example 15—3,4-Dihydroxybenzoic acid co-crystal form NF1 of S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

### Preparation Processes for the Mono 3,4-Dihydroxybenzoic Acid Co-Crystal Form NF1

Approximately 14 mg of the anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 0.7 mL acetone at 50° C. Approximately 10 mg of 3,4-dihydroxybenzoic acid were added and a cooling ramp was started three times (50-5° C. with 0.1 K/min). Since no precipitation was observed at 5° C., a vapour diffusion experiment was added by using n-pentane as anti-solvent. After 4 months, few particles were obtained.

NMR data for the mono 3,4-dihydroxybenzoic acid co-crystal form NF1

¹H NMR (500 MHz, DMSO-d6) δ 12.41-12.11 (m, 1H), 11.40-11.17 (m, 1H), 9.74-9.39 (m, 1H), 9.39-9.01 (m, 1H), 7.93 (s, 1H), 7.33 (d, J=2.1 Hz, 1H), 7.28 (dd, J=8.2, 2.1 Hz, 1H), 6.77 (d, J=8.3 Hz, 1H), 6.25-6.22 (m, 1H), 4.29 (q, J=2.8 Hz, 2H), 3.99 (s, 3H), 3.87 (t, J=5.4 Hz, 2H), 3.67-3.43 (m, 5H), 3.40 (d, J=11.2 Hz, 1H), 3.32 (d, J=11.2 Hz, 1H), 3.25-3.11 (m, 1H), 2.58-2.52 (m, 2H), 1.89-1.76 (m, 1H), 1.72-1.47 (m, 5H).

A powder X-ray diffraction pattern of the mono 3,4-dihydroxybenzoic acid co-crystal form NF1 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 28.

TABLE 13
Powder X-ray peak list of the mono 3,4-dihydroxybenzoic
acid co-crystal form NF1

| No. | °2θ (Cu—Kα₁ radiation) ± 0.2° |
|---|---|
| 1 | 7.3 |
| 2 | 10.8 |
| 3 | 13.9 |
| 4 | 15.2 |
| 5 | 15.9 |
| 6 | 16.5 |
| 7 | 17.4 |
| 8 | 18.0 |
| 9 | 18.6 |
| 10 | 19.3 |
| 11 | 20.2 |
| 12 | 21.3 |
| 13 | 23.0 |
| 14 | 23.5 |
| 15 | 25.1 |
| 16 | 25.8 |
| 17 | 27.2 |
| 18 | 28.2 |
| 19 | 29.5 |

Mono 3,4-dihydroxybenzoic acid co-crystal form NF1 is characterised by the following physical properties:
3,4-dihydroxybenzoic acid content (determined by ¹H-NMR spectroscopy) reveals 1.1 eq. 3,4-dihydroxybenzoic acid

### Example 16—Tartaric acid co-crystal form NF1 of S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

### Preparation Processes for the Tartaric Acid Co-Crystal Form NF1

Approximately 13 mg of anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 0.7 mL acetone at 50° C. Approximately 7 mg of tartaric acid were added, and a cooling ramp was started three times (50-5° C. with 0.1 K/min). Precipitation was observed at 5° C.

NMR data for the hemi tartaric acid co-crystal form NF1

¹H NMR (500 MHz, DMSO-d6): δ 12.73-12.53 (m, 1H), 11.35-11.23 (m, 1H), 7.94 (s, 1H), 6.25-6.22 (m, 1H), 5.13-4.93 (m, 1H), 4.31 (s, 1H), 4.29 (q, J=2.8 Hz, 2H), 4.00 (s, 3H), 3.87 (t, J=5.4 Hz, 2H), 3.66-3.43 (m, 5H), 3.41 (d, J=11.3 Hz, 1H), 3.32 (d, J=11.2 Hz, 1H), 3.26-3.12 (m, 1H), 2.58-2.53 (m, 2H), 1.91-1.75 (m, 1H), 1.73-1.47 (m, 5H).

A powder X-ray diffraction pattern of the hemi tartaric acid co-crystal form NF1 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 29.

TABLE 14
Powder X-ray peak list of the hemi
tartaric acid co-crystal form NF1

| No. | °2θ (Cu—Kα₁ radiation) ± 0.2° |
|---|---|
| 1 | 11.2 |
| 2 | 16.0 |
| 3 | 17.0 |
| 4 | 18.5 |
| 5 | 19.6 |
| 6 | 20.4 |
| 7 | 23.3 |
| 8 | 24.2 |
| 9 | 27.9 |

The hemi tartaric acid co-crystal form NF1 is characterised by the following physical properties:
Tartaric acid content (determined by ¹H-NMR spectroscopy) reveals 0.6 eq. tartaric acid
The thermal behaviour of the hemi tartaric acid co-crystal form NF1 shows a broad endothermic event <100° C., which goes along with weight loss step in the TGA profile (1.8% (w/w) up to −85° C.). This can be most likely assigned to release of water from weakly crystalline bulk phase. A broad melting/decomposition process can be observed at 13° C. (onset) in the DSC trace. DSC and TGA profiles are displayed in FIGS. 30A and 30B. The DSC scan of the hemi tartaric acid co-crystal form NF1 was acquired on a Mettler-Toledo DSC 821 with a heating rate of 5 K/min, using nitrogen purge gas at 50 mL/min. The TGA scan of the hemi tartaric acid co-crystal form NF1 was acquired on a Mettler-Toledo TGA 851 with a heating rate of 5 K/min, using nitrogen purge gas at 50 mL/min.

### Example 17—Tartaric acid co-crystal form NF2 of S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

### Preparation Processes for the Hemi Tartaric Acid Co-Crystal Form NF2

Approximately 67 mg of the anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 1.5 mL acetone at 50° C. Approximately 12 mg of tartaric acid were added, and a cooling ramp was started three times (50-5° C. with 0.1 K/min). Precipitation was observed at 5° C.

### NMR Data for the Hemi Tartaric Acid Co-Crystal Form NF2

¹H NMR (500 MHz, DMSO-d6) δ 13.11-12.14 (m, 1H), 11.29 (s, 1H), 7.93 (s, 1H), 6.25-6.22 (m, 1H), 5.24-4.70 (m, 1H), 4.31 (s, 1H), 4.29 (q, J=2.8 Hz, 2H), 3.99 (s, 3H), 3.87 (t, J=5.4 Hz, 2H), 3.68-3.45 (m, 5H), 3.40 (d, J=11.4 Hz, 1H), 3.33-3.29 (m, 1H), 3.26-3.09 (m, 1H), 2.58-2.52 (m, 2H), 1.89-1.75 (m, 1H), 1.72-1.46 (m, 5H).

A powder X-ray diffraction pattern of the hemi tartaric acid co-crystal form NF2 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 31.

TABLE 15
Powder X-ray peak list of the hemi
tartaric acid co-crystal form NF2

| No. | °2θ (Cu—Kα₁ radiation) ± 0.4° |
|---|---|
| 1 | 11.2 |
| 2 | 16.0 |
| 3 | 16.9 |
| 4 | 17.9 |
| 5 | 19.7 |
| 6 | 20.4 |
| 7 | 23.3 |
| 8 | 24.1 |
| 9 | 27.8 |

The hemi tartaric acid co-crystal form NF2 is characterised by the following physical properties:
Tartaric acid content (determined by ¹H-NMR spectroscopy reveals 0.5 eq. tartaric acid
Dissolution level of hemi tartaric acid co-crystal form NF2 in Fasted-State Simulated Intestinal Fluid [FaSSIF, pH 6.5] at 37° C. was determined to be approx. 145 μg/mL (after 30 min).

### Example 18—1,5-Naphthalenedisulfonic acid co-crystal form NF1 of S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

### Preparation Processes for the 1,5-Naphthalenedisulfonic Acid Co-Crystal Form NF1

Approximately 15 mg of the anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 0.7 mL acetone at 50° C. Approximately 12 mg of 1,5-naphthalenedisulfonic acid were added and a cooling ramp was started three times (50-5° C. with 0.1 K/min). Precipitation was observed at 5° C.

A powder X-ray diffraction pattern of the 1,5-naphthalenedisulfonic acid co-crystal form NF1 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 32.

TABLE 16
Powder X-ray peak list of the 1,5-naphthalenedisulfonic
acid co-crystal form NF1

| No. | °2θ (Cu—Kα₁ radiation) ± 0.4° |
|---|---|
| 1 | 6.5 |
| 2 | 8.4 |
| 3 | 10.1 |
| 4 | 10.4 |
| 5 | 12.0 |
| 6 | 13.1 |
| 7 | 14.8 |
| 8 | 15.3 |
| 9 | 16.9 |
| 10 | 17.7 |
| 11 | 18.2 |
| 12 | 18.6 |
| 13 | 19.4 |
| 14 | 20.3 |
| 15 | 21.0 |
| 16 | 21.8 |
| 17 | 22.2 |
| 18 | 22.8 |
| 19 | 25.0 |

The 1,5-naphthalenedisulfonic acid co-crystal form NF1 is characterised by the following physical properties:
The thermal behaviour of the 1,5-naphthalenedisulfonic acid co-crystal form NF1 shows no significant enthalpic events prior decomposition. In the temperature range up to 110° C., a weight loss of 2.2% (w/w) were observed, followed by a second weight loss step from 110-184° C. of 1.0% (w/w). The DSC and TGA profiles are displayed in FIGS. 33A and 33B. The DSC scan of form 1,5-naphthalenedisulfonic acid co-crystal form NF1 was acquired on a Mettler-Toledo DSC 821 with a heating rate of 5 K/min, using nitrogen purge gas at 50 mL/min. The TGA scan of 1,5-naphthalenedisulfonic acid co-crystal form NF1 was acquired on a Mettler-Toledo TGA 851 with a heating rate of 5 K/min, using nitrogen purge gas at 50 mL/min.

### Example 19—_{D}-Malic acid co-crystal form NF1 of S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide

### Preparation Processes for the Mono_{D}-Malic Acid Co-Crystal Form NF1

Approximately 12 mg of the anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide were dispersed in 0.5 mL acetone at 50° C. Approx. 8 mg of _{D}-malic acid were added, and a cooling ramp was started three times (50-5° C. with 0.1 K/min). Precipitation was observed at 5° C.

NMR data for the mono _{D}-Malic acid co-crystal form NF1

¹H NMR (500 MHz, DMSO-d6) δ12.57-12.11 (m, 2H), 11.30 (s, 1H), 7.94 (s, 1H), 6.26-6.22 (m, 1H), 5.49-5.29 (m, 1H), 4.29 (q, J=2.8 Hz, 2H), 4.26 (dd, J=7.7, 4.9 Hz, 1H), 4.00 (s, 3H), 3.87 (t, J=5.4 Hz, 2H), 3.66-3.43 (m, 5H), 3.41 (d, J=11.2 Hz, 1H), 3.32 (d, J=11.2 Hz, 1H), 3.25-3.12 (m, 1H), 2.61 (dd, J=15.6, 4.9 Hz, 1H), 2.58-2.53 (m, 2H), 2.44 (dd, J=15.6, 7.8 Hz, 1H), 1.89-1.74 (m, 1H), 1.74-1.46 (m, 5H).

A powder X-ray diffraction pattern of the mono _{D}-malic acid co-crystal form NF1 has been obtained by standard techniques as described in the European Pharmacopeia 6^{th} Edition chapter 2.9.33 and is characterised by the X-ray powder diffractogram (monochromatic Cu-Kα₁ radiation, λ=1.5406 Å, Stoe StadiP 611 KL transmission diffractometer) shown in FIG. 34.

TABLE 17
Powder X-ray peak list of the mono
_{D}-malic acid co-crystal form NF1

| No. | °2θ (Cu—Kα₁ radiation) ± 0.2° |
|---|---|
| 1 | 17.0 |
| 2 | 19.6 |
| 3 | 20.4 |
| 4 | 21.6 |
| 5 | 23.4 |
| 6 | 28.1 |

The mono _{D}-malic acid co-crystal form NF1 is characterised by the following physical properties:
Malic acid content (determined by 1H-NMR spectroscopy) reveals 1 eq. malic acid.
The thermal behaviour of mono _{D}-malic acid co-crystal form NF1 shows a very small endothermic event @˜137° C. This can be assigned to melting process of mono _{D}-malic acid co-crystal form NF1. In the temperature range up to 136° C., a weight loss of 2.8% (w/w) were observed. The DSC and TGA profiles are displayed in FIGS. 35A and 35B. The DSC scan of mono _{D}-Malic acid co-crystal form NF1 was acquired on a Mettler-Toledo DSC 821 with a heating rate of 5 K/min, using nitrogen purge gas at 50 mL/min. The TGA scan of mono _{D}-malic acid co-crystal form NF1 was acquired on a Mettler-Toledo TGA 851 with a heating rate of 5 K/min, using nitrogen purge gas at 50 mL/min.

### BRIEF DESCRIPTION OF THE FIGURES

FIG. 1 shows a powder X-ray diffractogram of the prior art amorphous state form of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide.

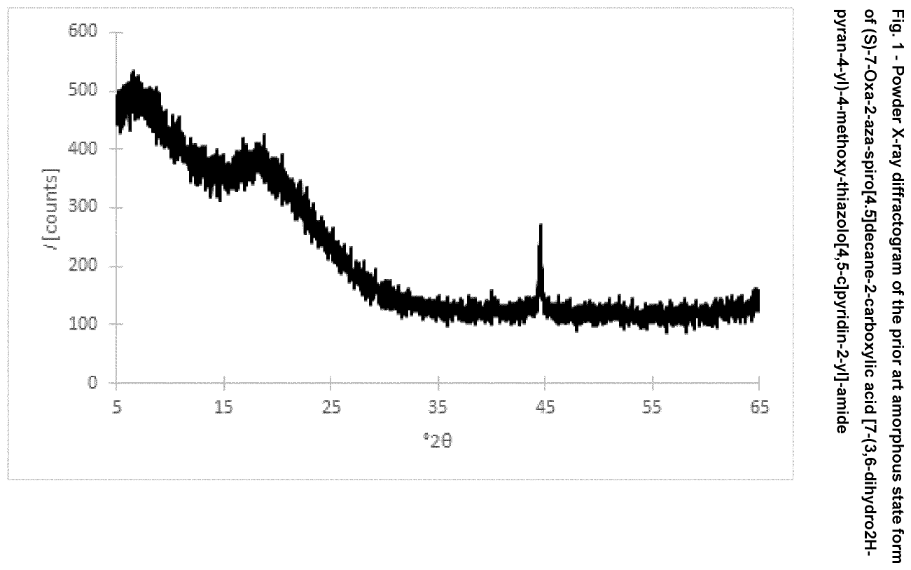

FIG. 2 shows a (2A) DSC scan (5 K/min) and a (2B) TGA scan (5 K/min) of amorphous state form) of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide.

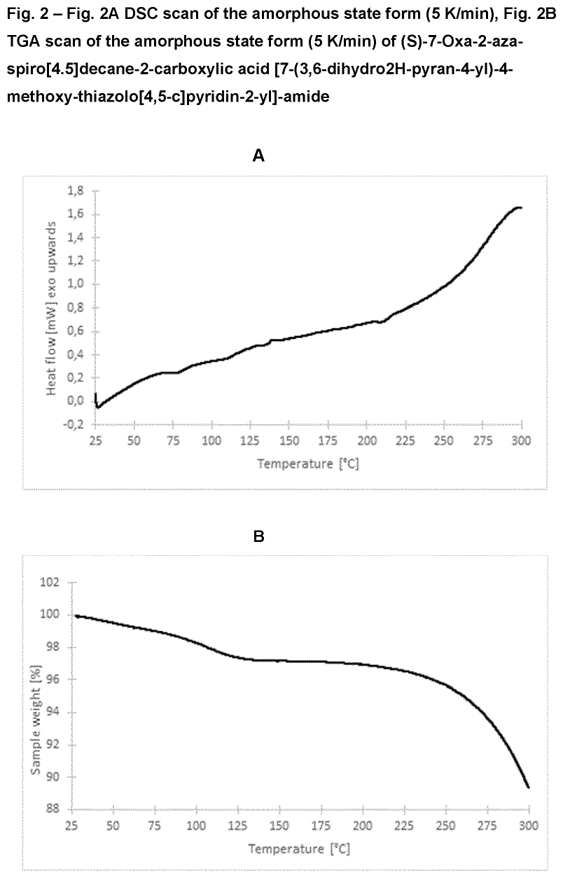

FIG. 3 shows a water vapour sorption isotherm (25° C.) of the amorphous state form of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide.

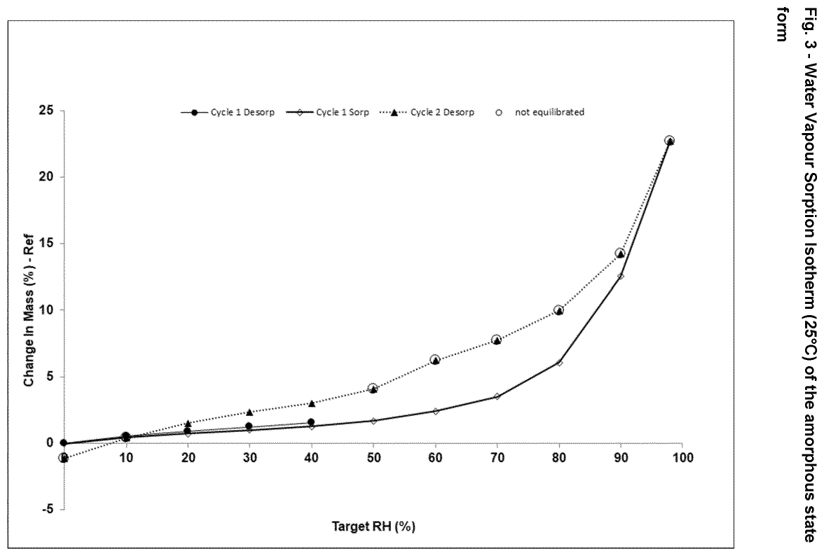

FIG. 4 shows a powder X-ray diffractogram of the crystalline anhydrous form A1 of (S)-7-Oxa-2-aza-spiro[4,5]decane-2-carboxylic acid [7-(3,6-dihydro2H-pyran-4-yl)-4-methoxy-thiazolo[4,5-c]pyridin-2-yl]-amide.

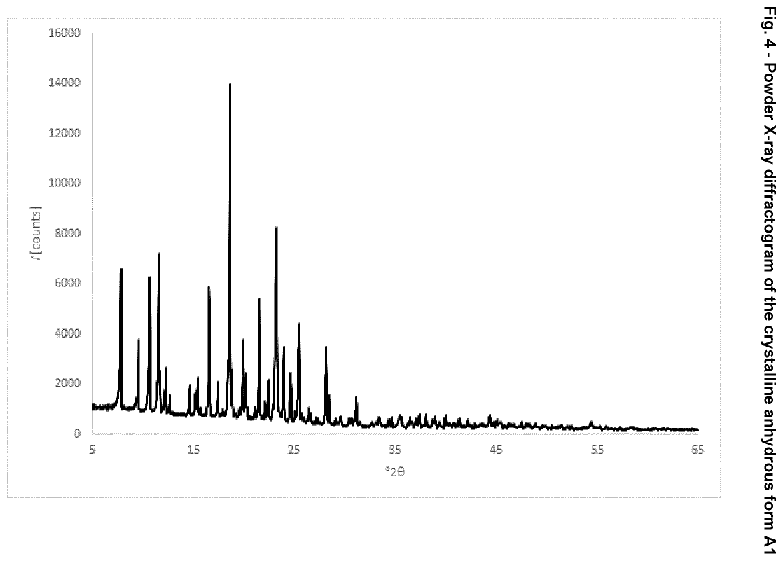

FIG. 5 shows the single crystal x-ray structure data of the crystalline anhydrous form A1.

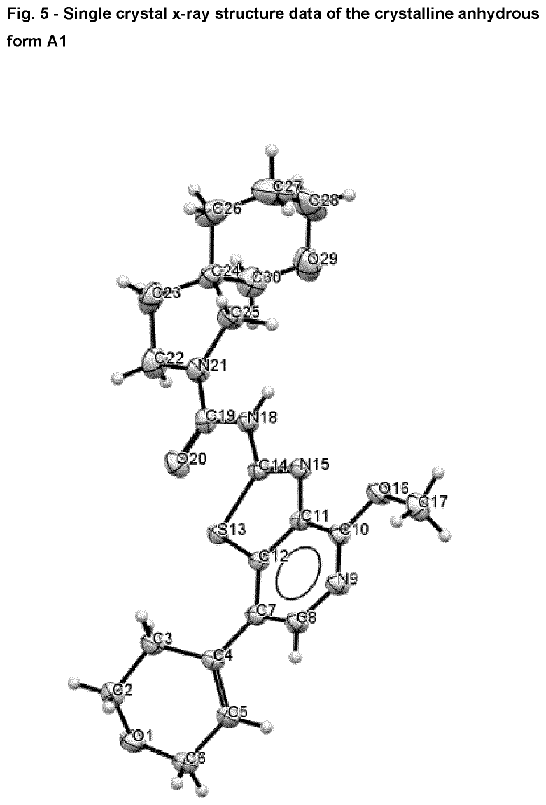

FIG. 6 shows a (6A) DSC scan of the crystalline anhydrous form A1 (5 K/min) and a (6B) TGA scan of the crystalline anhydrous form A1 (5 K/min).

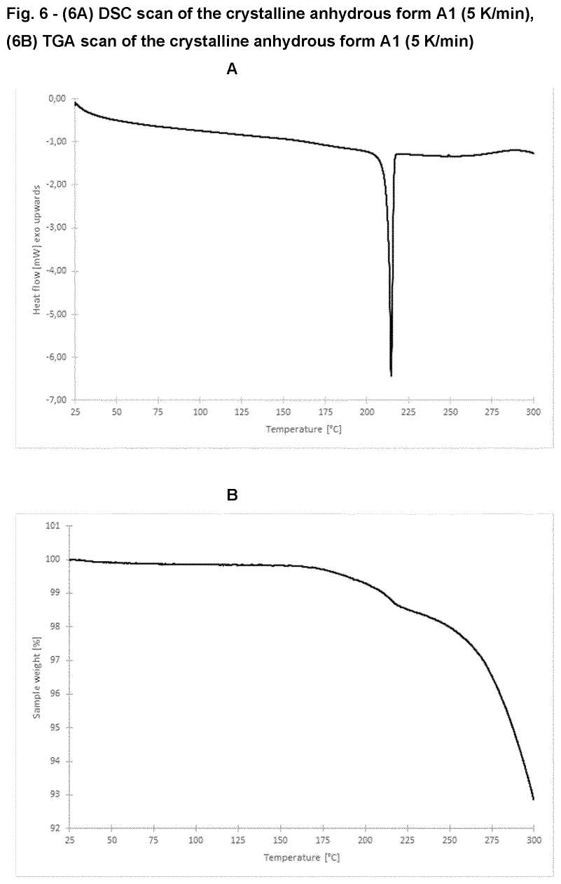

FIG. 7 shows a water vapour sorption isotherm (25° C.) of the crystalline anhydrous form A1.

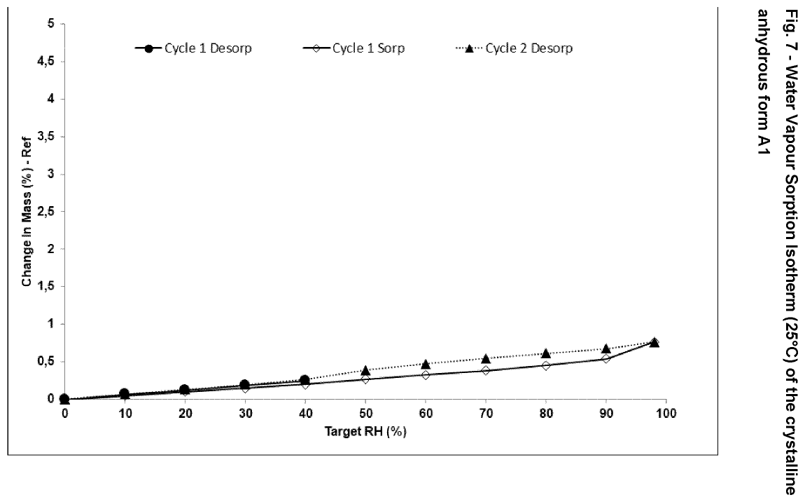

FIG. 8 shows a powder X-ray diffractogram of the hydrate form NF2.

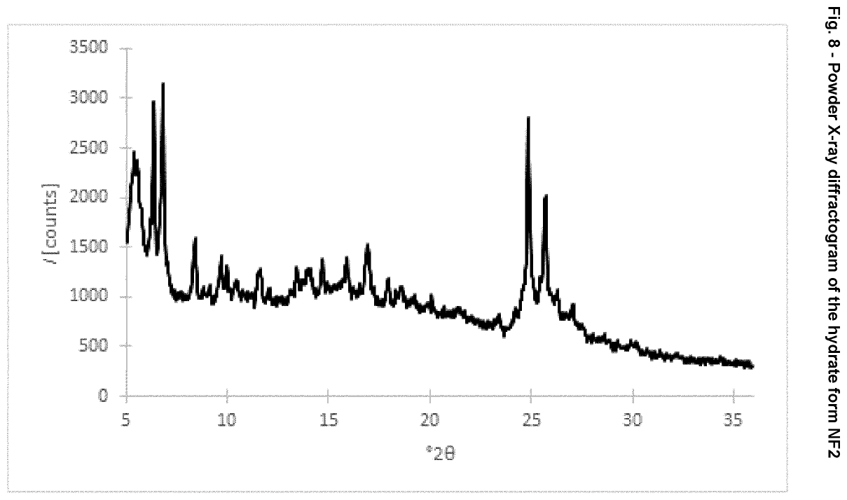

FIG. 9 shows a powder X-ray diffractogram of the hydrate form NF3.

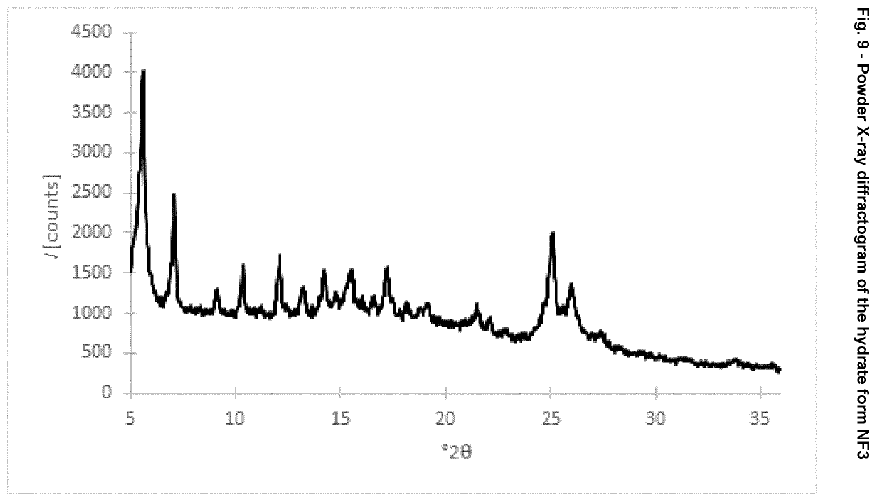

FIG. 10 shows a powder X-ray diffractogram of the hydrate form NF4.

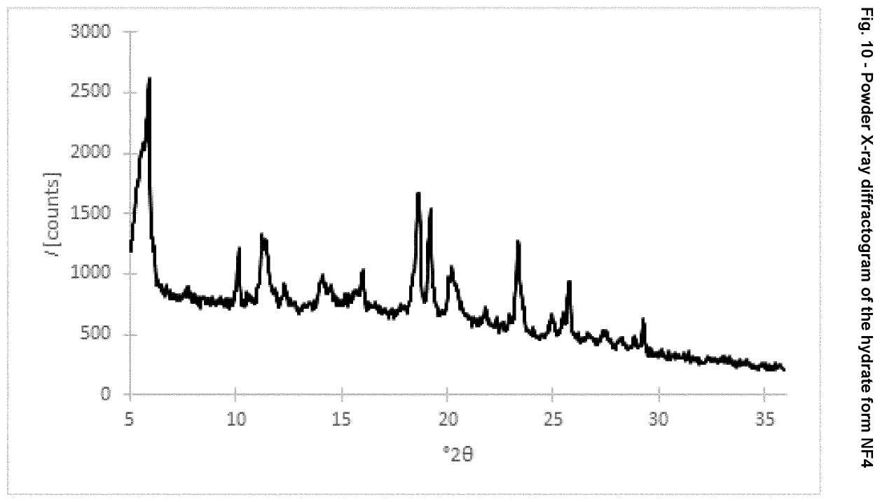

FIG. 11 shows a powder X-ray diffractogram of the hydrate form NF12.

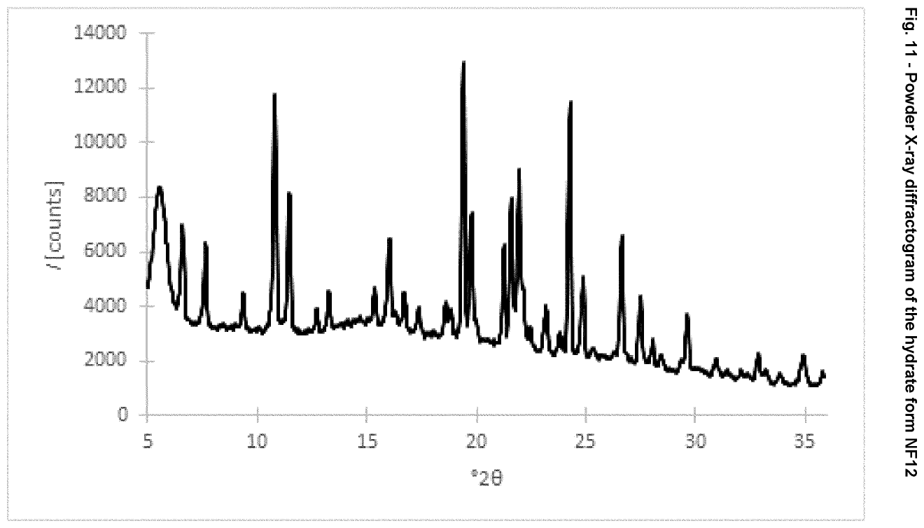

FIG. 12 shows a powder X-ray diffractogram of the mono fumaric acid co-crystal form A1.

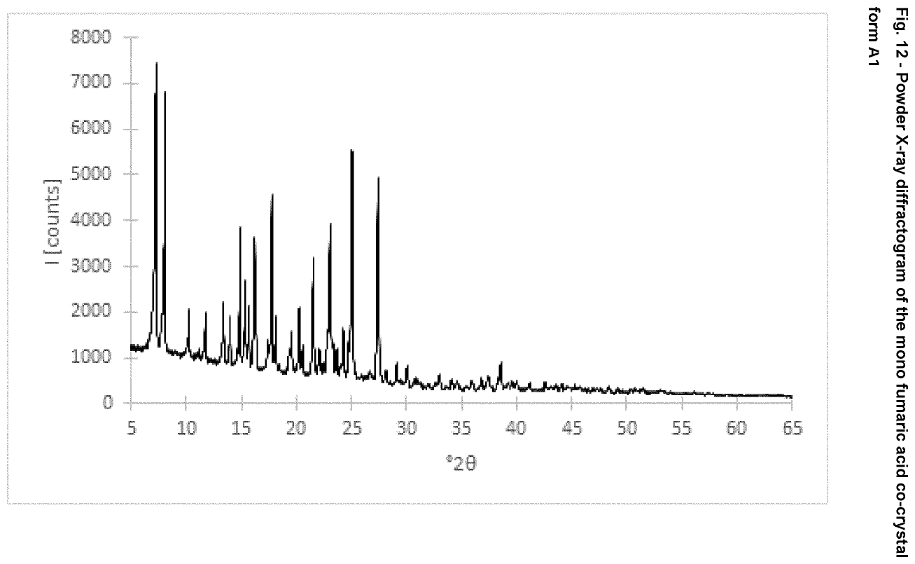

FIG. 13 shows a single crystal structure of the mono fumaric acid co-crystal form A1.

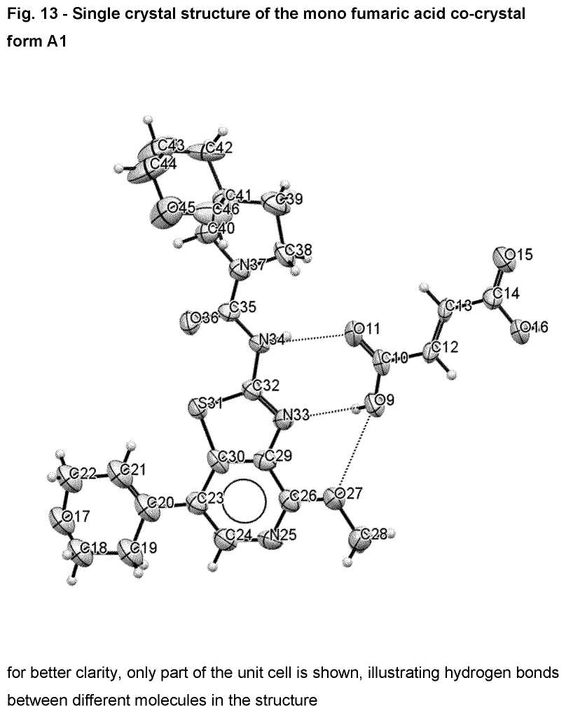

FIG. 14 shows a (14A) DSC scan of the mono fumaric acid co-crystal form A1 (5 K/min) and a (14B) TGA scan of the mono fumaric acid co-crystal form A1 (5 K/min).

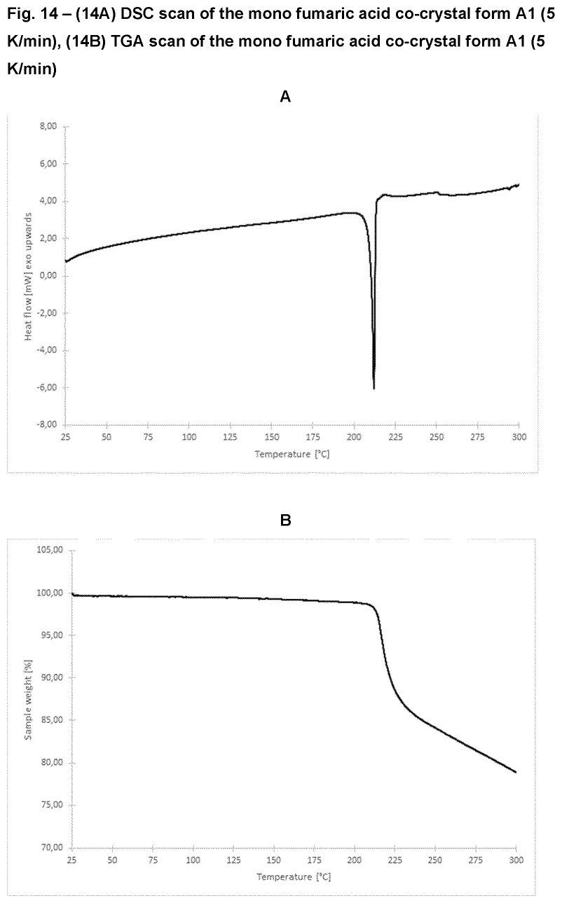

FIG. 15 shows water vapour sorption isotherm (25° C.) of the mono fumaric acid co-crystal form A1.

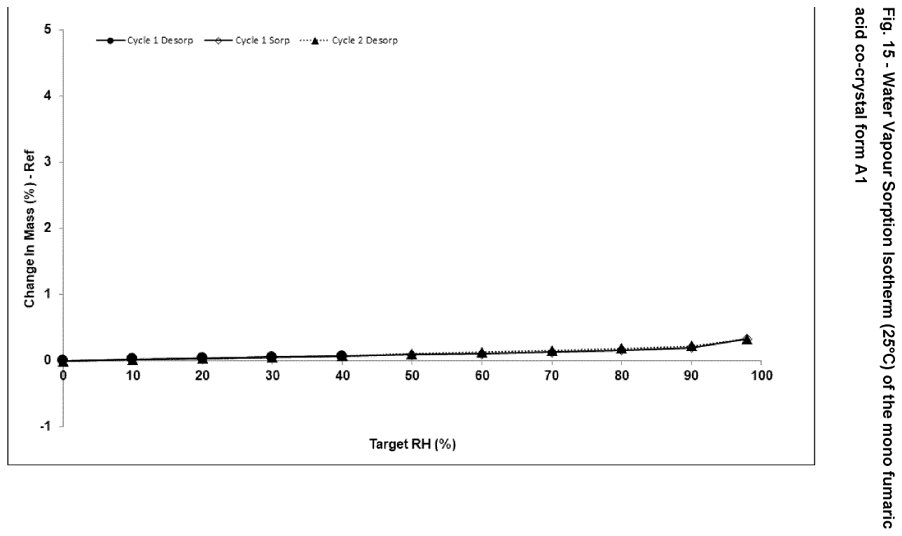

FIG. 16 shows a powder X-ray diffractogram of the hemi fumaric acid co-crystal form A2.

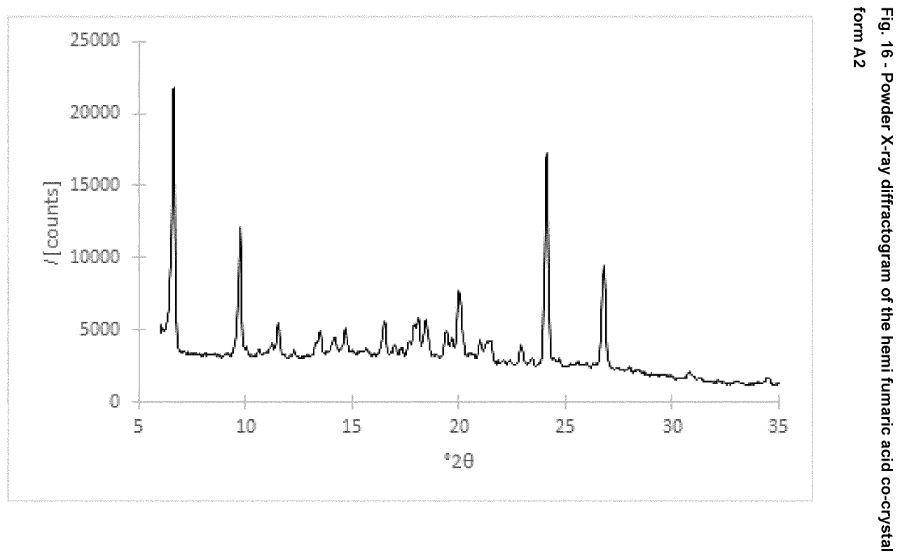

FIG. 17 shows the single crystal structure of the hemi fumaric acid co-crystal form A2.

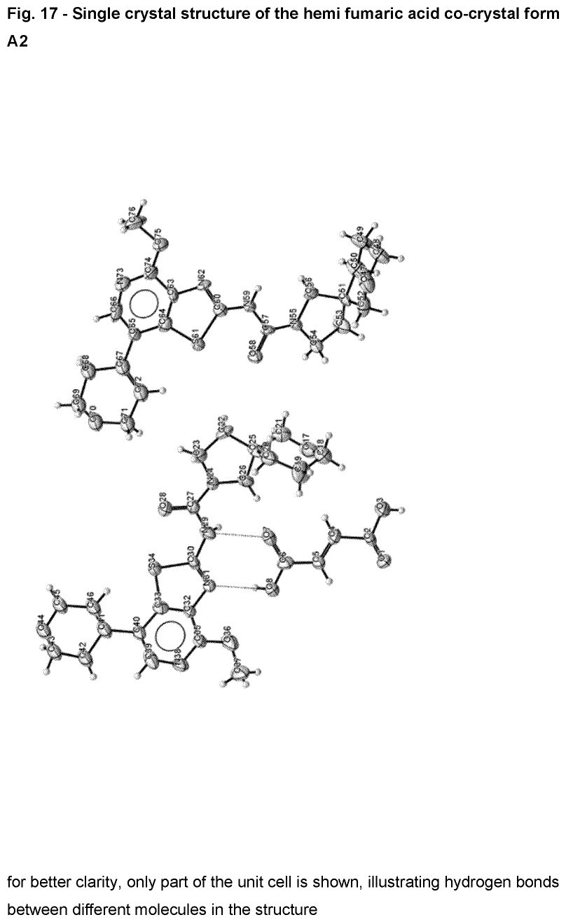

FIG. 18 shows a (18A) DSC scan of the hemi fumaric acid co-crystal form A2 (5 K/min) and a (18B) TGA scan of the hemi fumaric acid co-crystal form A2 (5 K/min).

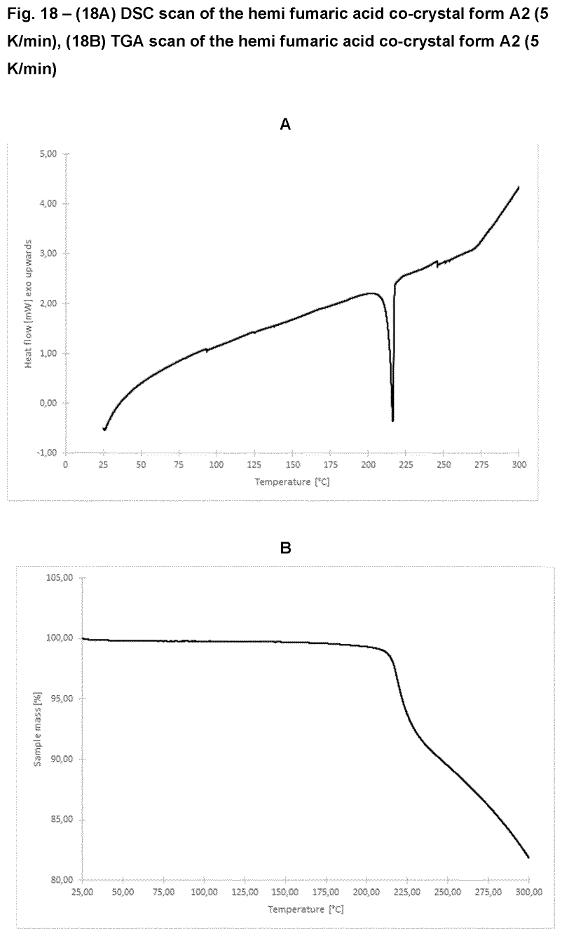

FIG. 19 shows a water vapour sorption isotherm (25° C.) of the hemi fumaric acid co-crystal form A2.

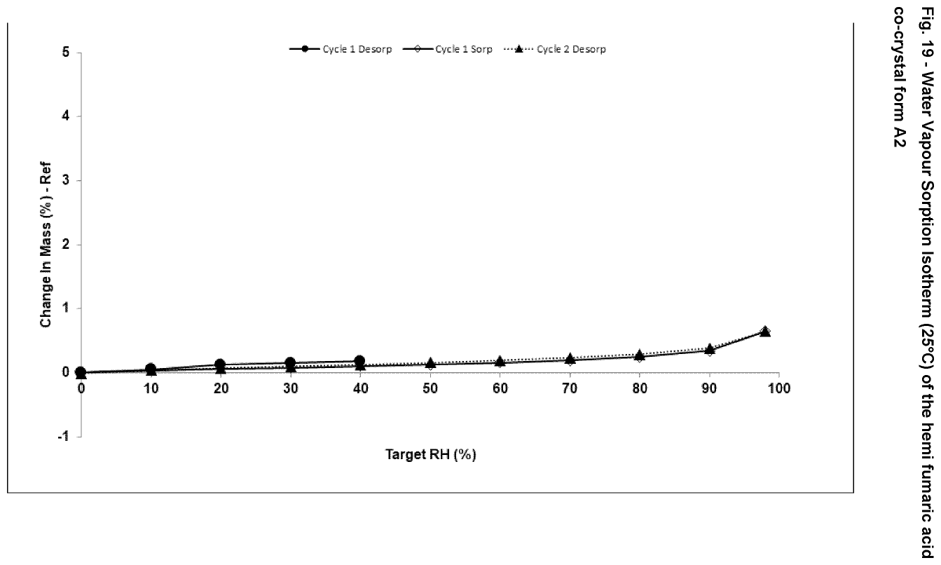

FIG. 20 shows a powder X-ray diffractogram of the hemi fumaric acid co-crystal form NF1.

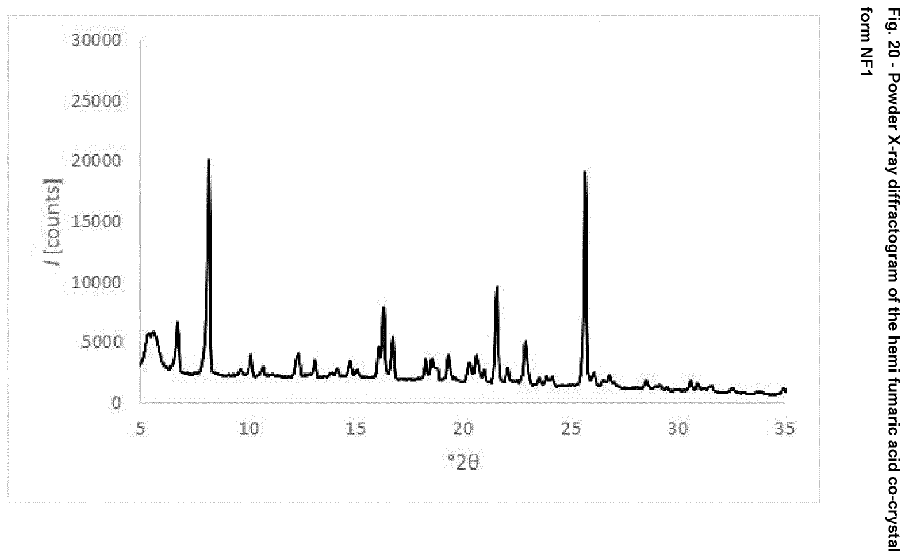

FIG. 21 shows a (21A) DSC scan of the hemi fumaric acid co-crystal form NF1 (10 K/min) and a (21B) TGA scan of the hemi fumaric acid co-crystal form NF1 (10 K/min).

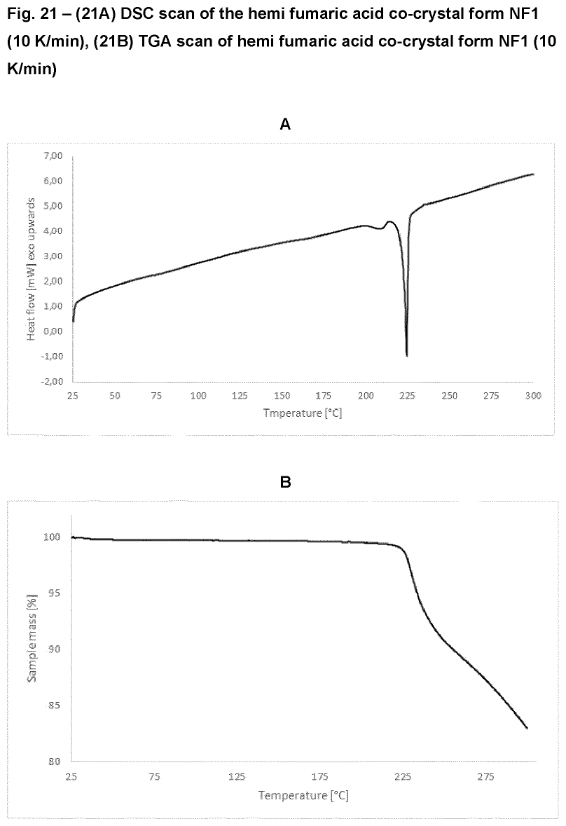

FIG. 22 shows a powder X-ray diffractogram of the hemi fumaric acid co-crystal form NF2.

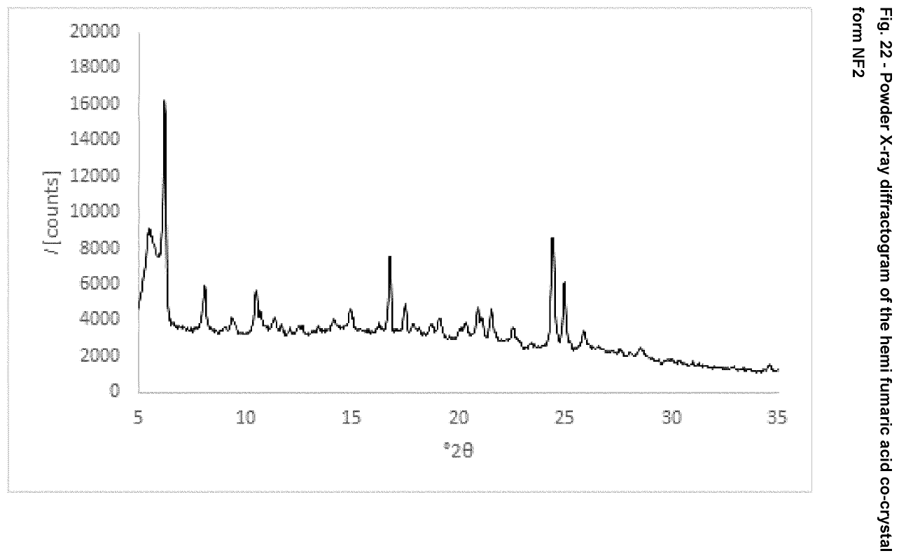

FIG. 23 shows a (23A) DSC scan of the hemi fumaric acid co-crystal form NF2 (5 K/min) and a (23B) TGA scan of hemi fumaric acid co-crystal form NF2 (5 K/min).

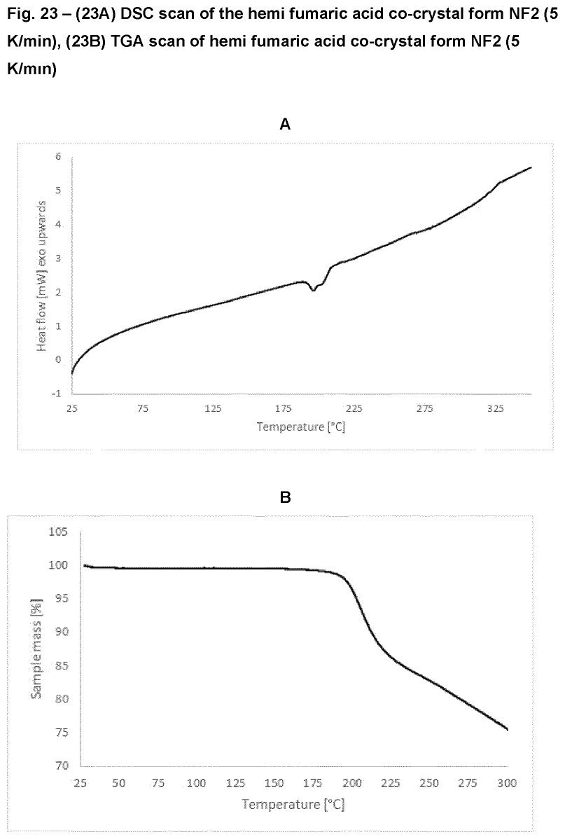

FIG. 24 shows a powder X-ray diffractogram of the mono 3-hydroxybenzoic acid co-crystal form NF1.

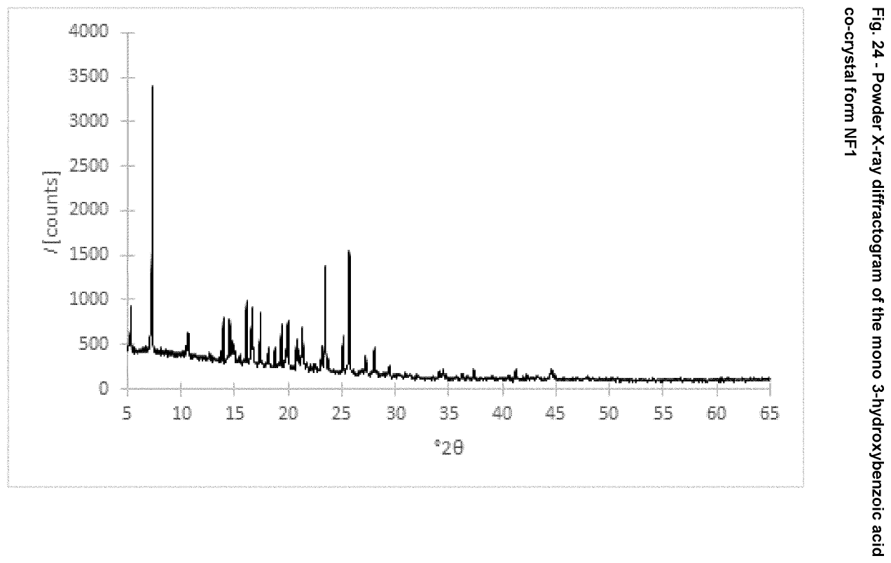

FIG. 25 shows a (25A) DSC scan of th mono 3-hydroxybenzoic co-crystal form NF1 (5 K/min) and a (25B) TGA scan of the mono 3-hydroxybenzoic co-crystal form NF1 (5 K/min).

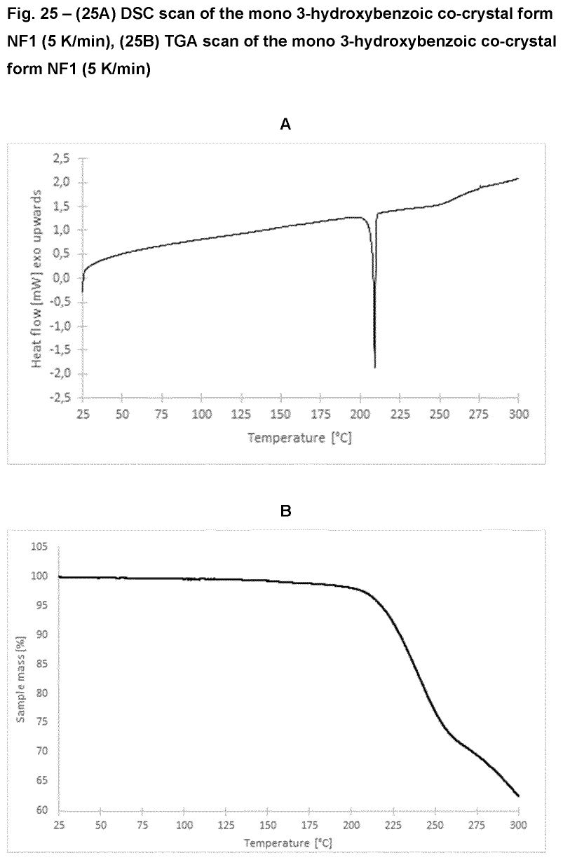

FIG. 26 shows a water vapour sorption isotherm (25° C.) of the mono 3-hydroxybenzoic co-crystal form NF1.

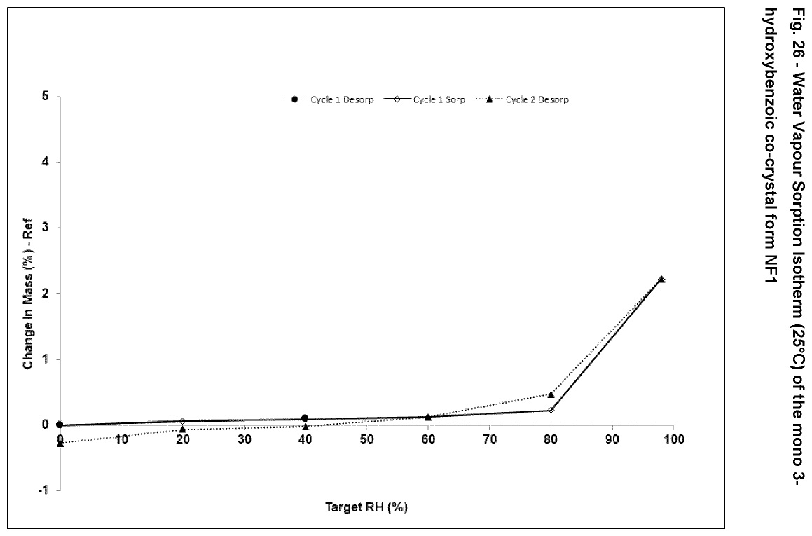

FIG. 27 shows a powder X-ray diffractogram of the mono 3-hydroxybenzoic co-crystal form NF2.

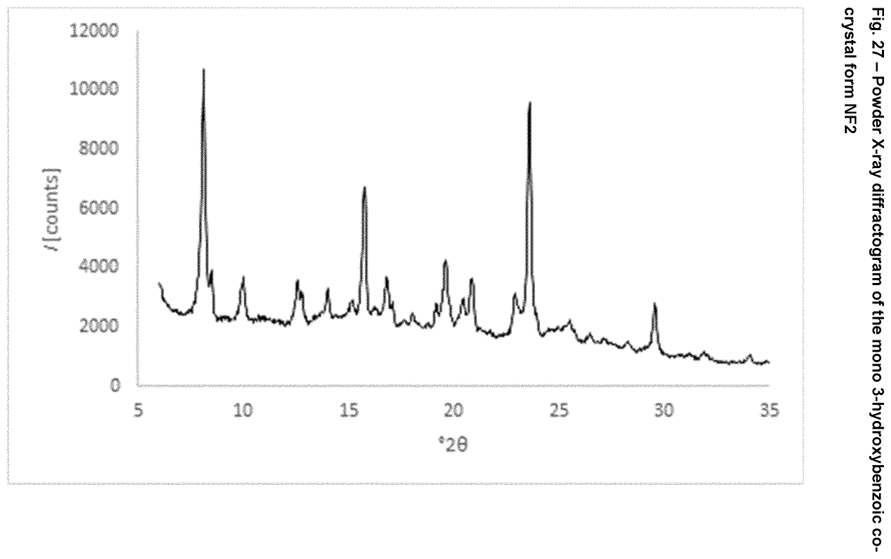

FIG. 28 shows a powder X-ray diffractogram of the mono 3,4-dihydroxybenzoic acid co-crystal form NF1.

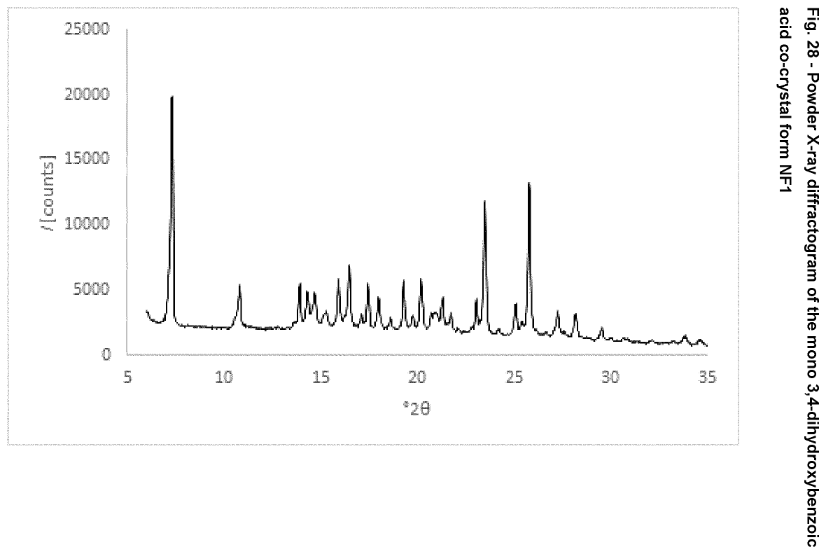

FIG. 29 shows a powder X-ray diffractogram of the hemi tartaric acid co-crystal form NF1.

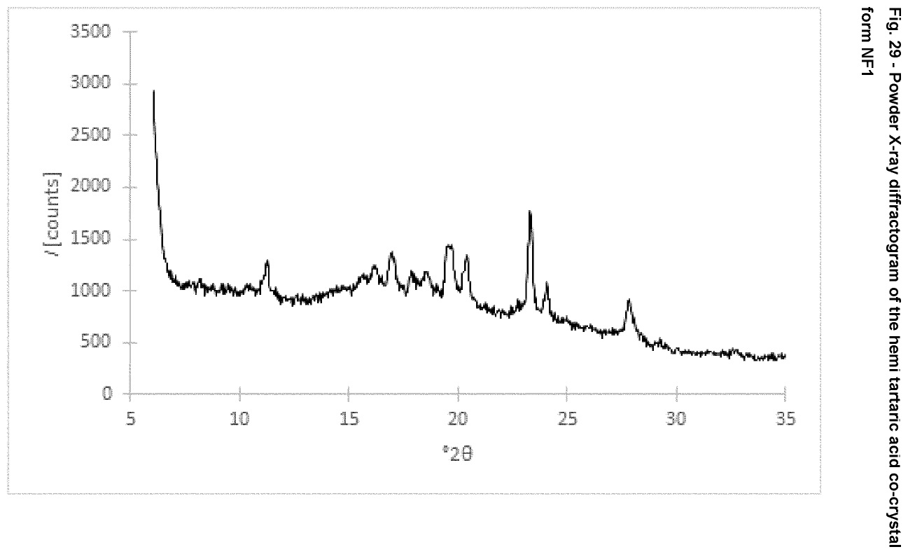

FIG. 30 shows a (30A) DSC scan of the hemi tartaric acid co-crystal form NF1 (5 K/min) and a (30B) TGA scan of the hemi tartaric acid co-crystal form NF1 (5 K/min).

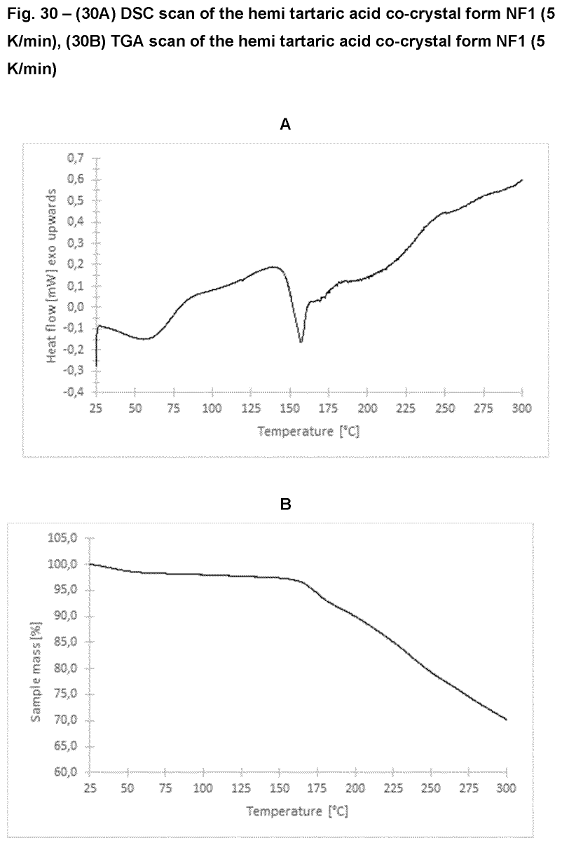

FIG. 31 shows a powder X-ray diffractogram of the hemi tartaric acid co-crystal form NF2.

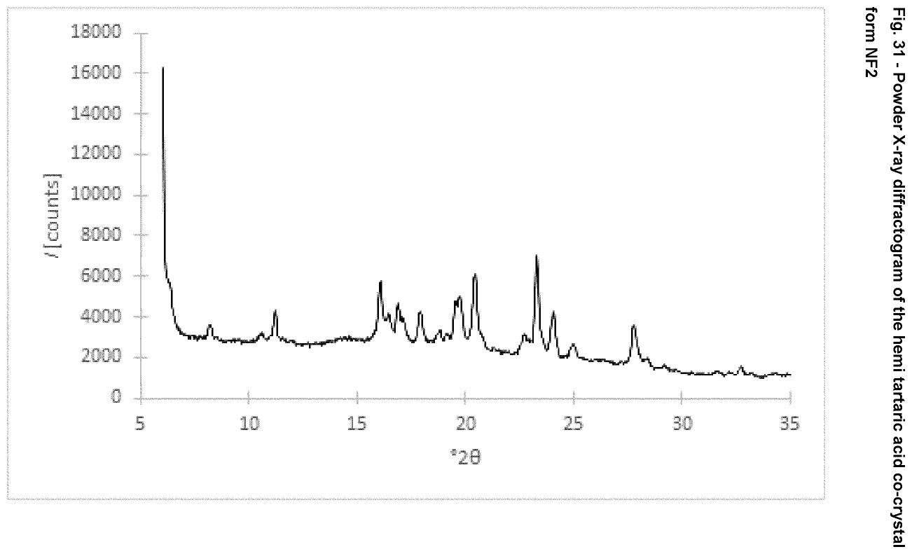

FIG. 32 shows a powder X-ray diffractogram of the 1,5-naphthalenedisulfonic acid co-crystal form NF1.

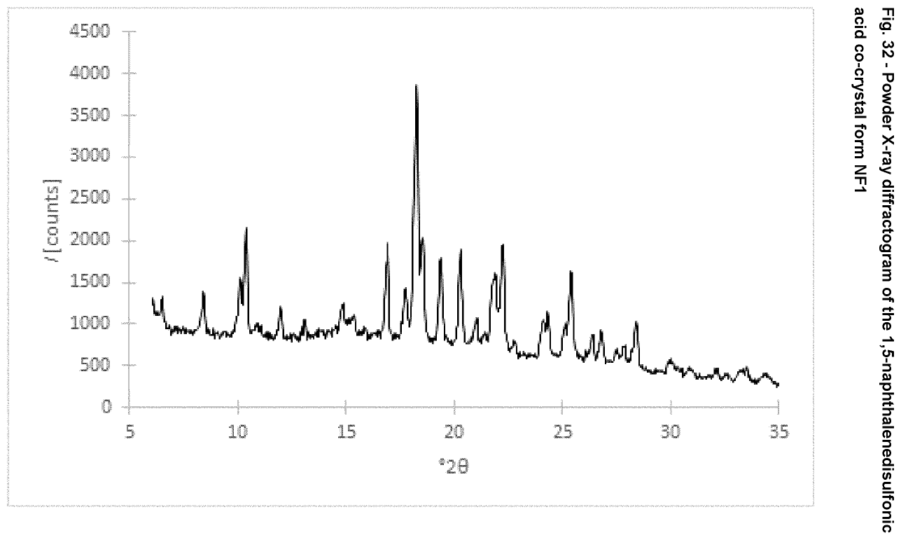

FIG. 33 shows a (33A) DSC scan of the 1,5-naphthalenedisulfonic acid co-crystal form NF1 and a (33B) TGA scan of the 1,5-naphthalenedisulfonic acid co-crystal form NF1 (5 K/min).

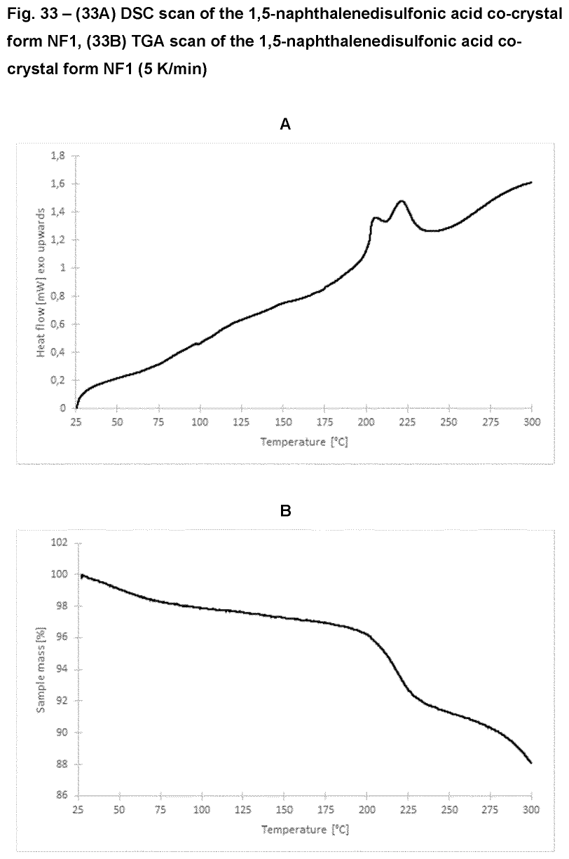

FIG. 34 shows a powder X-ray diffractogram of the mono _{D}-malic acid co-crystal form NF1.

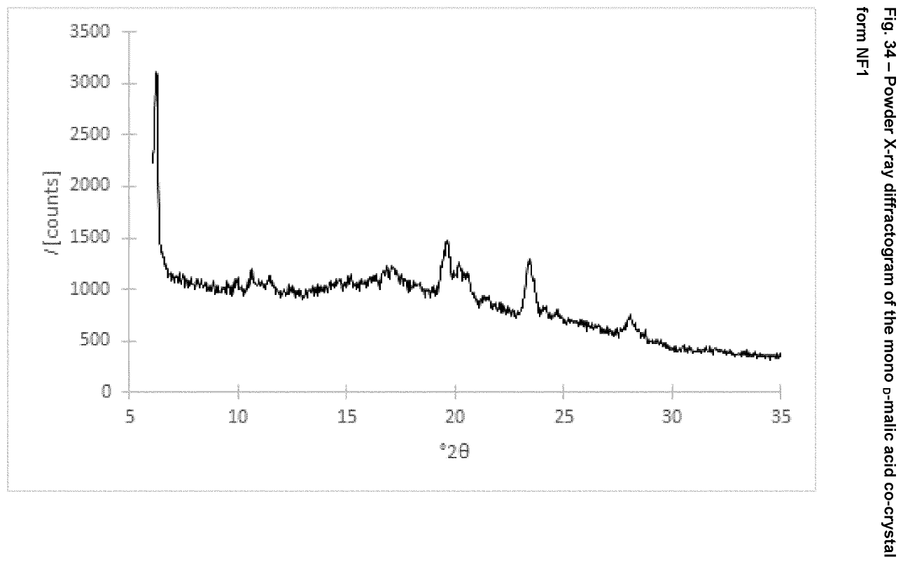

FIG. 35 shows a (35A) DSC scan of the mono _{D}-malic acid co-crystal form NF1 and a (35B) TGA scan of mono _{D}-malic acid co-crystal form NF1.

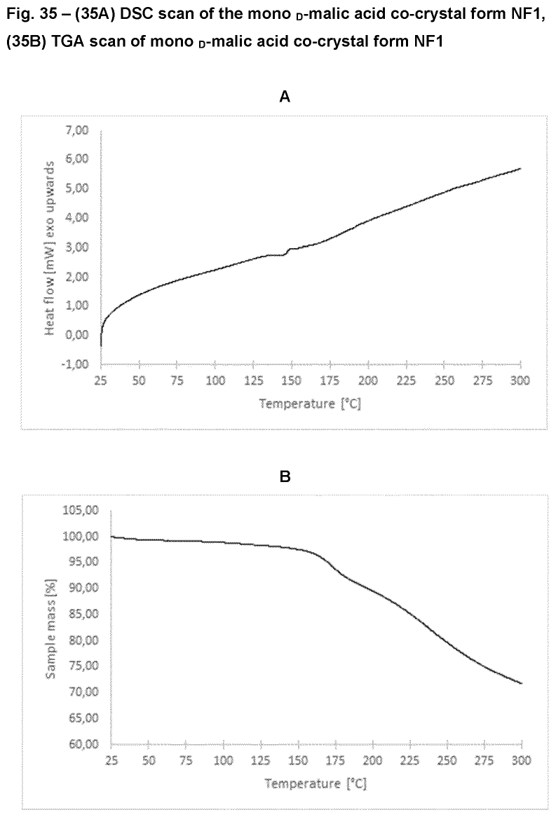

1. A crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide.

2. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 1, wherein the
crystalline form is a crystalline anhydrous form A1.

3. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 1, wherein the
crystalline form is selected from the group consisting of the hydrate forms NF2, NF3, NF4 and NF12.

4. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl-amide according to claim 1, wherein the
crystalline form is a co-crystal form selected from the group consisting of
a) a mono fumaric acid co-crystal form A1,
b) a hemi fumaric acid co-crystal form A2,
c) a hemi fumaric acid co-crystal form NF1,
d) a hemi fumaric acid co-crystal form NF2,
e) a mono 3-hydroxybenzoic acid co-crystal form NF1,
f) a mono 3-hydroxybenzoic acid co-crystal form NF2,
g) a hemi tartaric acid co-crystal form NF1,
h) a hemi tartaric acid co-crystal form NF2,
i) a mono _{D}-malic acid co-crystal form NF1,
j) a 1,5-naphthalenedisulfonic acid co-crystal form NF1 and
k) a mono 3,4-dihydroxybenzoic acid co-crystal form NF1.

5. The crystalline anhydrous form A1 of (S)-7-oxa-2-aza-spiro[4,5]decane-2-carboxylic acid 17-(3,6-dihydro-2H-pyran-4-yl)-4-methoxy-thiazolo[4,5-c]pyridin-2-yl]-amide according to claim 2,
wherein the crystalline anhydrous form A1 has the characteristic peaks:

| No. | °2θ (Cu—Kα₁ radiation) ± 0.2° |
|---|---|
| 1 | 7.8 |
| 2 | 9.5 |
| 3 | 10.6 |
| 4 | 11.6 |
| 5 | 12.2 |
| 6 | 15.4 |
| 7 | 16.5 |
| 8 | 18.6 |
| 9 | 19.9 |
| 10 | 20.2 |
| 11 | 21.5 |
| 12 | 22.4 |
| 13 | 23.2 |
| 14 | 23.9 |
| 15 | 24.6 |
| 16 | 25.5 |
| 17 | 28.1 |
| 18 | 28.5 |

6. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the mono fumaric acid co-crystal form A1 has the characteristic peaks:

| No. | °2θ (Cu—Kα₁ radiation) ± 0.2° |
|---|---|
| 1 | 7.8 |
| 2 | 9.5 |
| 3 | 10.6 |
| 4 | 11.6 |
| 5 | 12.2 |
| 6 | 15.4 |
| 7 | 16.5 |
| 8 | 18.6 |
| 9 | 19.9 |
| 10 | 20.2 |
| 11 | 21.5 |
| 12 | 22.4 |
| 13 | 23.2 |
| 14 | 23.9 |
| 15 | 24.6 |
| 16 | 25.5 |
| 17 | 28.1 |
| 18 | 28.5 |

7. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the hemi fumaric acid co-crystal form A2 has the characteristic peaks:

| No. | °2θ (Cu—Kα₁ radiation) ± 0.2° |
|---|---|
| 1 | 6.6 |
| 2 | 9.7 |
| 3 | 11.5 |
| 4 | 13.4 |
| 5 | 14.1 |
| 6 | 14.7 |
| 7 | 16.5 |
| 8 | 18.0 |
| 9 | 18.5 |
| 10 | 19.4 |
| 11 | 20.0 |
| 12 | 21.4 |
| 13 | 22.9 |
| 14 | 24.1 |
| 15 | 26.8 |

8. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the hemi fumaric acid co-crystal form NF1 has the characteristic peaks:

| No. | °2θ (Cu—Kα₁ radiation) ± 0.2° |
|---|---|
| 1 | 6.7 |
| 2 | 8.1 |
| 3 | 10.1 |
| 4 | 12.3 |
| 5 | 16.1 |
| 6 | 16.3 |
| 7 | 16.7 |
| 8 | 19.3 |
| 9 | 21.5 |
| 10 | 22.9 |
| 11 | 25.7 |

9. The crystalline form of (S)-7-oxa-2-aza-spiro[4.5]decane-2-carboxylic acid [7-(3,6-dihydro-2H-pyran-4-yl)-4-methoxy-thiazolo[4,5-c]-pyridin-2-yl]-amide according to claim 4, wherein the hemi fumaric acid co-crystal form NF2 has the characteristic peaks:

| No. | °2θ (Cu—Kα₁ radiation) ± 0.2° |
|---|---|
| 1 | 6.2 |
| 2 | 8.1 |
| 3 | 9.4 |
| 4 | 10.5 |
| 5 | 11.3 |
| 6 | 14.2 |
| 7 | 14.9 |
| 8 | 16.8 |
| 9 | 17.5 |
| 10 | 19.1 |
| 11 | 20.3 |
| 12 | 20.9 |
| 13 | 21.6 |
| 14 | 22.6 |
| 15 | 24.4 |
| 16 | 25.9 |

10. The clystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the mono 3-hydroxybenzoic acid co-crystal form NF1 has the characteristic peaks:

| No. | °2θ (Cu—Kα₁ radiation) ± 0.2° |
|---|---|
| 1 | 7.3 |
| 2 | 10.6 |
| 3 | 13.9 |
| 4 | 14.5 |
| 5 | 16.1 |
| 6 | 16.6 |
| 7 | 17.4 |
| 8 | 18.1 |
| 9 | 18.7 |
| 10 | 19.4 |
| 11 | 20.0 |
| 12 | 20.8 |
| 13 | 21.3 |
| 14 | 23.1 |
| 15 | 23.5 |
| 16 | 25.1 |
| 17 | 25.7 |
| 18 | 27.2 |
| 19 | 28.1 |

11. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the mono 3-hydroxybenzoic acid co-crystal form NF2 has the characteristic peaks:

| No. | °2θ (Cu—Kα₁ radiation) ± 0.2° |
|---|---|
| 1 | 8.1 |
| 2 | 8.5 |
| 3 | 10.0 |
| 4 | 12.6 |
| 5 | 14.0 |
| 6 | 15.7 |
| 7 | 16.8 |
| 8 | 19.6 |
| 9 | 20.4 |
| 10 | 20.9 |
| 11 | 22.9 |
| 12 | 23.6 |
| 13 | 29.5 |

12. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the mono 3,4-dihydroxybenzoic acid co-crystal form NF1 has the characteristic peaks:

| No. | °2θ (Cu—Kα₁ radiation) ± 0.2° |
|---|---|
| 1 | 7.3 |
| 2 | 10.8 |
| 3 | 13.9 |
| 4 | 15.2 |
| 5 | 15.9 |
| 6 | 16.5 |
| 7 | 17.4 |
| 8 | 18.0 |
| 9 | 18.6 |
| 10 | 19.3 |
| 11 | 20.2 |
| 12 | 21.3 |
| 13 | 23.0 |
| 14 | 23.5 |
| 15 | 25.1 |
| 16 | 25.8 |
| 17 | 27.2 |
| 18 | 28.2 |
| 19 | 29.5 |

13. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the hemi tartaric acid co-crystal form NF1 has the characteristic peaks:

| No. | °2θ (Cu—Kα₁ radiation) ± 0.2° |
|---|---|
| 1 | 11.2 |
| 2 | 16.0 |
| 3 | 17.0 |
| 4 | 18.5 |
| 5 | 19.6 |
| 6 | 20.4 |
| 7 | 23.3 |
| 8 | 24.2 |
| 9 | 27.9 |

14. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the hemi tartaric acid co-crystal form NF1 has the characteristic peaks:

| No. | °2θ (Cu—Kα₁ radiation) ± 0.2° |
|---|---|
| 1 | 11.2 |
| 2 | 16.0 |
| 3 | 16.9 |
| 4 | 17.9 |
| 5 | 19.7 |
| 6 | 20.4 |
| 7 | 23.3 |
| 8 | 24.1 |
| 9 | 27.8 |

15. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the 1,5-naphthalenedisulfonic acid co-crystal form NF1 has the characteristic peaks:

| No. | °2θ (Cu—Kα₁ radiation) ± 0.2° |
|---|---|
| 1 | 6.5 |
| 2 | 8.4 |
| 3 | 10.1 |
| 4 | 10.4 |
| 5 | 12.0 |
| 6 | 13.1 |
| 7 | 14.8 |
| 8 | 15.3 |
| 9 | 16.9 |
| 10 | 17.7 |
| 11 | 18.2 |
| 12 | 18.6 |
| 13 | 19.4 |
| 14 | 20.3 |
| 15 | 21.0 |
| 16 | 21.8 |
| 17 | 22.2 |
| 18 | 22.8 |
| 19 | 25.0 |

16. The crystalline form of (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide according to claim 4, wherein the mono _{D}-malic acid co-crystal form NF1 has the characteristic peaks:

| No. | °2θ (Cu—Kα₁ radiation) ± 0.2° |
|---|---|
| 1 | 17.0 |
| 2 | 19.6 |
| 3 | 20.4 |
| 4 | 21.6 |
| 5 | 23.4 |
| 6 | 28.1 |

17. A (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide containing one or more of the crystalline form according to claim 1.

18. The (S)-7-oxa-2-aza-spiro[4,5]decane-2-carboxylic acid [7-(3,6-dihydro-2H-pyran-4-yl)-4-methoxy-thiazolo[4,5-c]pyridin-2-yl]-amide essentially consisting of one or more of the crystalline form according to claim 1.

19. A method for the preparation of the crystalline form according to claim 4, the method comprising:
suspending (S)-7-oxa-2-aza-spiro[4.5]decane-2-carboxylic acid [7-(3,6-dihydro-2H-pyran-4-yl)-4-methoxy-thiazolo[4,5-c]pyridin-2-yl]-amide in an organic solvent at elevated temperature,
adding the equivalent amount of a co-former, and
carrying out a cooling crystallization.

20. A medicament, comprising:
one or more of the crystalline form according to claim 1 or mixtures thereof in all ratios for the treatment and/or prophylaxis of cancer.

21. A pharmaceutical preparation, comprising:
one or more of the crystalline form according to claim 1 or mixtures thereof in all ratios and optionally further excipients and/or adjuvants.

22. A process for the preparation of a pharmaceutical preparation, the process comprising:
bringing that one or more of the crystalline form according to claim 1 or mixtures thereof in all ratios into a suitable dosage form together with a solid, liquid or semi-liquid excipient or adjuvant.
